(19) United States
(12) Patent Application Publication
(10) Pub. No.: US 2021/0252151 A1
(43) Pub. Date: Aug. 19, 2021

(54) METHOD FOR OPTICAL OPENING OF THE BLOOD-BRAIN BARRIER

(71) Applicant: BOARD OF REGENTS, THE UNIVERSITY OF TEXAS SYSTEM, Austin, TX (US)

(72) Inventors: Zhenpeng QIN, Allen, TX (US); Xiaoqing LI, Richardson, TX (US); Xiuying LI, Plano, TX (US); Shashank SIRSI, Dallas, TX (US); Heather HAYENGA, Dallas, TX (US); Edward PAN, Dallas, TX (US); Robert BACHOO, Dallas, TX (US)

(73) Assignee: BOARD OF REGENTS, THE UNIVERSITY OF TEXAS SYSTEM, Austin, TX (US)

(21) Appl. No.: 17/251,977

(22) PCT Filed: Jun. 14, 2019
(86) PCT No.: PCT/US2019/037172

§ 371 Date: Dec. 14, 2020

Related U.S. Application Data
(60) Provisional application No. 62/685,910, filed on Jun. 15, 2018.

(51) Int. Cl. A61K 41/00; A61N 5/06; A61K 45/06; A61K 51/10; A61K 49/00; A61K 47/68

(52) U.S. Cl. CPC A61K 41/0028; A61N 5/062; A61K 45/06; A61N 2005/067; A61K 49/0002; A61K 47/6843; A61K 51/1093

(57) ABSTRACT

The blood-brain barrier (BBB) excludes most drugs and poses a significant challenge to treat brain diseases. Current methods for BBB opening yield modest outcomes in clinical applications due to safety and toxicity. This disclosure relates to methods of optically opening the BBB by laser excitation of tight-junction targeted nanoparticles to induce BBB transient opening. The excitation of plasmonic nanoparticles produces localized effects such as nanoscale heating and photomechanical force leading to BBB transiently opening to allow macromolecules across it. The safe and predictable platform for brain drug delivery will improve therapies for brain disease such as cancers, infections and neurologic disorders.

### PRIORITY CLAIM

This application claims benefit of priority to U.S. Provisional Application Ser. No. 62/685,910, filed Jun. 15, 2018, the entire contents of which are hereby incorporated by reference.

### BACKGROUND

### 1. Field

The present disclosure relates generally to the fields of cell biology, neurobiology and membrane physiology. More particularly, it concerns methods for transiently opening of the blood-brain barrier (BBB) by optically exciting light-absorbing particles to locally disrupt BBB tight junctions, permitting drugs to pass into the brain.

### 2. Related Art

Brain homeostasis is critical and necessary for neurons communication because neurons can sense subtle change in their microenvironment. To address these requirements, blood vessels in the brain have the blood-brain barrier (BBB) to keep normal brain function¹. The BBB is a highly selective physical barrier that only allow passage of small gaseous molecules (such as O₂, CO₂), small number of hydrophobic molecules, and those that have specific transporters, including sugar and some amino acids (leucine and valine)². Generally, only molecules below 500 Da can pass through the BBB^{3,4}. Among the molecular compositions of the BBB, the tight junction (TJ), a belt-like adhesive region between adjacent endothelial cells, poses a significant structure barrier for BBB by sealing the paracellular space between the endothelial cells. The existence of BBB is an effective way to protect brain from common infections including antibody and toxins present in blood. However, it is also the major reason for low accumulation of therapeutic drugs in the brain, such as in the treatment of central nervous system (CNS) diseases, including brain tumors. It has been reported that over 98% of drugs do not show useful activity in the CNS⁴.

Various strategies are being developed to overcome the BBB transport issue and enhance the concentration of therapeutic drugs in the brain. Current techniques for BBB disruption include osmotic disruption⁵, MRI-guide focus ultrasound BBB disruption⁶⁻⁸, vasoactive agent disruption⁹. The hypertonic chemical mannitol introduces osmotic pressure between endothelial cells and disrupts TJs by shrinking the endothelial cells to increase the BBB permeability. However, osmotically opening BBB leads to significant neurotoxicity, including epileptic seizures. Recently, the focused ultrasound (FUS) has been reported to effectively open BBB due to thermal coagulative effect or mechanical wave via coupling with microbubbles, but there remains a concern that fluid leakage associated with mechanical stretch injury to the brain microcirculation and sterile inflammatory response. Lastly, BBB opening by vasoactive agents activates receptors on endothelial cell to increase the permeability. Unfortunately, the vasoactive agents activate the blood vessels in the entire brain and even in the periphery, thus inducing non-specific increase of permeability. Therefore, there is great need for an accurate and effective method to open the BBB temporarily and safely for the treatment of CNS disorders.

### SUMMARY

Thus, in accordance with the present disclosure, there is provided a method of generating nanoscale heating and photomechanical effects in vivo at the blood-brain barrier of a subject comprising (a) administering to said subject a light-absorbing particle, such as a plasmonic noble metal nanoparticle, wherein said light-absorbing particle comprises a targeting agent that directs said light-absorbing particle to the blood-brain barrier; and (b) contacting said light-absorbing particle with a short pulse laser signal. The light-absorbing particle may be administered systemically, such as intravenously, intra-arterially, intrathecally, or retro-orbitally.

The short pulse laser signal may be a femtosecond, a picosecond or a nanosecond laser signal. The targeting agent may be a receptor ligand, a receptor ligand mimic, or an antibody. The targeting agent may target said light-absorbing particle to a blood-brain barrier tight junction, or endothelial membrane receptor such as transferrin. The targeting agent may target JAM-A, Claudin-5, ZO-1, or transferrin. The subject may be a human or a non-human animal.

The method may further comprise administering to said subject a therapeutic or diagnostic agent. The therapeutic or diagnostic agent may be attached to said light-absorbing particle. The therapeutic or diagnostic agent may not be attached to said light-absorbing particle. The laser signal may be a near-infrared signal or a or visible signal.

In another embodiment, there is provided a method of delivering an agent in vivo to the brain of a subject comprising (a) administering to said subject a light-absorbing particle, wherein said light-absorbing particle comprises a targeting agent that directs said light-absorbing particle to the blood-brain barrier; (b) administering to said subject an agent; and (c) contacting said light-absorbing particle with a short pulse laser signal. The agent may be a diagnostic agent, such as a dye, a fluorophore, and chromophore, a contrast agent, or a radionuclide, or a therapeutic agent, such as an anti-cancer agent (e.g., a chemotherapeutic, a radiotherapeutic, an immunotherapeutic, or a gene therapeutic) or an antibiotic, an antifungal or an antiviral. The therapeutic agent may also be a neurotherapeutic, such as an anti-dementia drug, an anti-neurodegenerative disease drug, anti-edema agent such as glyburide, or a gene therapy agent such as AAV.

The targeting agent may target JAM-A, Claudin-5, ZO-1, or endothelial membrane receptor such as transferrin. The subject may be a human or a non-human animal. The agent may be attached to said light-absorbing particle or may not be attached to said light-absorbing particle. The laser signal may be a near-infrared signal or a visible signal. The light-absorbing particle and/or said agent may be administered systemically, such as intravenously, intra-arterially, intrathecally, or retro-orbitally.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the disclosure, and vice versa. Furthermore, compositions and kits of the disclosure can be used to achieve methods of the disclosure.

Throughout this application, the term “about” is used to indicate that a value includes the inherent variation of error for the device, the method being employed to determine the value, or the variation that exists among the study subjects.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device or a method that “comprises,” “has,” “contains,” or “includes” one or more elements possesses those one or more elements but is not limited to possessing only those one or more elements or steps. Likewise, an element of a device or method that “comprises,” “has,” “contains,” or “includes” one or more features possesses those one or more features but is not limited to possessing only those one or more features.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed.

FIG. 1. Concept for optical BBB transient opening after laser excite vasculature-targeting plasmonic gold nanoparticle. In step 2, JAM-A antibody is omitted for nano-sonication visual.

FIG. 2. Co-localization of TJ-targeting AuNP and TJ. JAM-A is displayed in green and nuclei are red. Green signal on the borders of cells indicate JAM-A expression on hCMEC/D3 cell membrane. Scale bar, 10 μm.

FIG. 3. Schematic of measurement of TEER and permeability of BBB monolayer in vitro model.

FIG. 4. Normalized TEER and apparent permeability (Pₐₚₚ) of 40 kDa FITC-dextran of hCMEC/D3 monolayer treated by 45 nm AuNP-BV16 and laser pulses (35.78 mJ/cm², 5 Hz).

FIG. 5. Normalized TEER of D3 monolayer treated by 45 nm AuNP-BV16 after laser irradiation with different energies (50 pulses, 5 Hz)

FIG. 6. Normalized TEER of D3 monolayer treated by 45 nm AuNP-BV16 after laser irradiation with different frequencies (35.78 mJ/cm², 50 pulses)

FIG. 7. Cell viability measurement post laser irradiation (25 pulses with 5, 10, 25 and 35 mJ/cm²). The student's T test shows no significant difference between laser excitation and control group.

FIG. 8. Monolayer resistance measurement with half laser irradiation. The lower resistance of the monolayer with 50% laser irradiation (35 mJ/cm², 10 pulses) than prediction indicates the occurrence of BBB opening propagation.

FIG. 9. Calcium response post ps laser irradiation (35 mJ/cm², 1 pulse). Scale bar: 100 μm.

FIGS. 10A-C. In vivo biodistribution measurement of TJ-targeting AuNP. (FIG. 10A) ICP-MS analysis of AuNP-BV11 distribution in major organs, % ID/g: % injection dose/gram. (FIG. 10B) Biodistribution comparison between targeting (AuNP-BV11) and non-targeting (AuNP-PEG) gold nanoparticles. (FIG. 10C) Pharmacokinetics of AuNP-BV11 and AuNP-PEG in mouse.

FIG. 11. Silver enhancement staining shows microscopic distribution of AuNP in major organs. Scale bar: 20 μm.

FIGS. 12A-B. Toxicity measurement of TJ-targeting AuNP. (FIG. 12A) Measurement of body weight loss. AuNP-BV11: 36.9 μg/g. Saline: 100 (FIG. 12B) H&E staining of the brain at day 24 post injection of AuNP-BV11 and AuNP-PEG.

FIGS. 13A-B. Optical BBB temporary opening in vivo. (FIG. 13A) Schematic of BBB opening in vivo. (FIG. 13B) Effect of tight junction targeting on BBB opening (1 pulse, 25 mJ/cm2). Right: AuNP-BV11 targeting to TJ along BBB; Left: AuNP-PEG without targeting function. Scale bar, 4 mm.

FIG. 14. The time window effect on BBB opening after AuNP Injection. Laser irradiation at 1, 6 12 and 24 hours (1 pulse, 25 mJ/cm²) post AuNP-BV11 injection, followed by Evans blue (EB) injection. Scale bar, 4 mm.

FIGS. 15A-B. Effect of laser parameters on BBB opening. (FIG. 15A) Effect of laser pulse energy on BBB opening (1 pulse). (FIG. 15B) Effect of laser pulse number on BBB opening (25 mJ/cm²). Scale bar, 4 mm.

FIGS. 16A-D. Monte Carlo analysis. (FIG. 16A) Fluorescent images of brain tissue with various laser pulse energy. The bulk orange color on the top the coronal section indicates Evans blue extravasation. Scale bar: 2 mm. (FIG. 16B) The fluence distribution inside brain tissue. (FIG. 16C) The average threshold simulation. (FIG. 15D) Light penetration and Evans blue penetration with different laser fluence.

FIGS. 17A-C. Reversibility of BBB opening with various energy (1 pulse). (FIG. 17A) Schematic of BBB recovery study. Evans blue injection at 0 hr, 1 hr, 6 hr, day 3 post laser irradiation. (FIG. 17B-C) BBB recovery with low and high laser pulse energy. Scale bar: 4 mm.

FIGS. 18A-B. BBB opening with high spatial resolution by optical control (1 pulse, 25 mJ/cm²). (FIG. 18A) Laser beam size of 1, 2, 3 is 0.8 mm, 0.8 mm, 2.5 mm by pinhole, respectively. (FIG. 18B) BBB opening in mouse thalamus with optical fiber.

FIG. 19. Brain vasculatures analysis of optical BBB opening (1 pulse, 25 mJ/cm²) on right hemisphere.

FIGS. 20A-B. Brain cells response to transcranial optical BBB opening (25 mJ/cm², 1 pulse). (FIG. 20A) Immunohistochemistry (IHC) staining of neurons. Purple: NeuN labels cell body of neurons, Green: Neurofilament heavy polypeptide (NEFH) labels the dendrites of neurons, Orange: Lectin labels the blood vessels, blue: DAPI labels the nuclei. (FIG. 20B) IHC staining of astrocytes. Purple: GFAP indicate active astrocytes, orange: lectin label cerebral vessels.

FIGS. 21A-B. Transmission electron microscopy (TEM) of the brain ultrastructures with laser BBB opening (5 mJ/cm², 1 pulse, 6 hours). The tight junction (TJ), pericyte (asterisk), basement membrane (BM) appear intact with laser treatment in FIG. 21B, compared with control group without laser treatment in FIG. 21A, while edema was observed in the BBB opening group 6 hours after laser treatment; The synapse (label “S”) and mitochondria (label “MT”) seem normal in FIG. 21A and FIG. 21B. L: lumen. Scale bar: 1 μm.

FIGS. 22A-C. Delivery of functional molecules (25 mJ/cm², 1 pulse on right hemisphere). (FIG. 22A) Anti-cancer drug: doxorubicin (Dox). Red signal indicates Dox extravasation after laser application (Figure B) Endogenous IgG: mouse. Bulk purple signal suggests mouse IgG extravasation. (FIG. 22C) Exogenous IgG: Human. Bulk green signal indicates human IgG extravasation.

FIG. 23. Exemplary workflow for an embodiment of the disclosure.

FIG. 24. Exemplary workflow for an embodiment of the disclosure.

FIG. 25. Exemplary workflow for an embodiment of the disclosure.

FIG. 26. Exemplary workflow for an embodiment of the disclosure.

FIGS. 27A-C. Characterization of BBB in vitro model by (FIG. 27A) Transendothelial electrical resistance (TEER), (FIG. 27B) apparent permeability coefficient (Papp), (FIG. 27C) Immunocytochemistry (ICC) staining of JAM-A. Scale bar, 10 μm.

FIGS. 28A-F. Characterization of TJ-targeting AuNP by (FIGS. 28A-D): Dynamic light scattering (DLS), (FIG. 28E): UV-visual spectroscopy, (FIG. 28F): Transmission electron microscopy (TEM).

FIG. 29. Co-localization of TJ-targeting AuNP and TJ with various AuNP concentration. JAM-A is displayed in green and nuclei are red. Top: 2.9 nM, Middle: 0.5 nM, bottom: 0.05 nM. Green signal on the borders between cells indicates JAM-A expression on cell membrane. Scale bar, 10 μm.

FIG. 30. Normalized TEER value of hCMEC/D3 monolayer treated by laser pulse alone (35.8 mJ/cm², 5 Hz).

FIG. 31. Normalized TEER value of hCMEC/D3 monolayer treated by laser pulse alone (35.8 mJ/cm², 5 Hz).

FIG. 32. Normalized TEER of hCMEC/D3 monolayer treated by 0.5 nM 60 nm AuNP-BV16 after laser irradiation with various pulses (24.4 mJ/cm², 5 Hz). The TEER recover to 100% in 6 hours.

FIG. 33. Normalized TEER of hCMEC/D3 monolayer treated by 0.5 nM 60 nm GNP-BV16 after laser irradiation with different energy (25 pulses, 5 Hz). The TEER recover to 100% in 6 hours.

FIG. 34. Normalized TEER of hCMEC/D3 monolayer treated by 0.5 nM 60 nm GNP-BV16 after laser irradiation with various frequency (24.8 mJ/cm², 25 pulses). The TEER recover to 100% in 6 hours.

FIG. 35. Apparent permeability of 40 kDa FITC-dextran of hCMEC/D3 monolayer after laser irradiation (24.8 mJ/cm², 5 Hz).

FIG. 36. Pathology analysis of major organs using H&E staining.

FIG. 37. Evans blue extravasation with low laser pulse energy (1 pulse). The bulk yellow signal on top left and right indicates the extravasation of Evans blue post laser irradiation. Left hemisphere: 2.5 mJ/cm², right hemisphere: 5 mJ/cm². Scale bar: 5 mm.

FIG. 38. Evans blue extravasation with medium and high laser pulse energy (1 pulse). The bulk yellow signal on top left and right indicates the extravasation of Evans blue post laser irradiation. Left hemisphere: 10 mJ/cm², right hemisphere: 25 mJ/cm². Scale bar: 5 mm.

FIGS. 39A-C. Endogenous mouse IgG extravasation after BBB opening (25 mJ/cm², 1 pulse). (FIG. 39A) The endogenous mouse IgG leak into brain tissue at 0.5 hour post-laser irradiation. The purple signal of coronal section indicated the extravasation of mouse IgG. (FIG. 39B) Control: No purple signal (mouse IgG) was observed when the brain tissue was not incubated with Donkey-anti-mouse IgG antibody (0.5 hour). (FIG. 39C) No endogenous mouse IgG leak into brain tissue when BBB recover at day 3.

FIG. 40. Human IgG extravasation with medium and high laser pulse energy (1 pulse). The bulk green signal (arrow) on to left and right of coronal section indicated the extravasation of human IgG post laser irradiation. Left hemisphere: 10 mJ/cm², right hemisphere: 25 mJ/cm². Scale bar: 5 mm.

FIGS. 41A-C. Blood-brain barrier opening with nanosecond laser pulse and AuNP. (FIG. 41A) Cell viability tests demonstrates the laser and nanoparticle treatment don't affect endothelial viability; (FIG. 41B) Relative TEER changes; (FIG. 41C) Permeability to macromolecules (FD-4 refers to 4 kDa FITC-dextran).

### DETAILED DESCRIPTION

It has been reported that optical excitation of light-absorbing particles generates localized effects on surrounding biomolecules including protein denaturation and photomechanical effect. When plasmonic gold nanoparticles (AuNPs) are locally overheated with a short laser pulse, this generates a series of thermophysical effects including local heating, acoustic signal, and in extreme cases nucleation of a very thin volume (nanometer size) of water in the immediate surrounding medium. This localized effect could perturb the protein and cell function without injuring the cells.

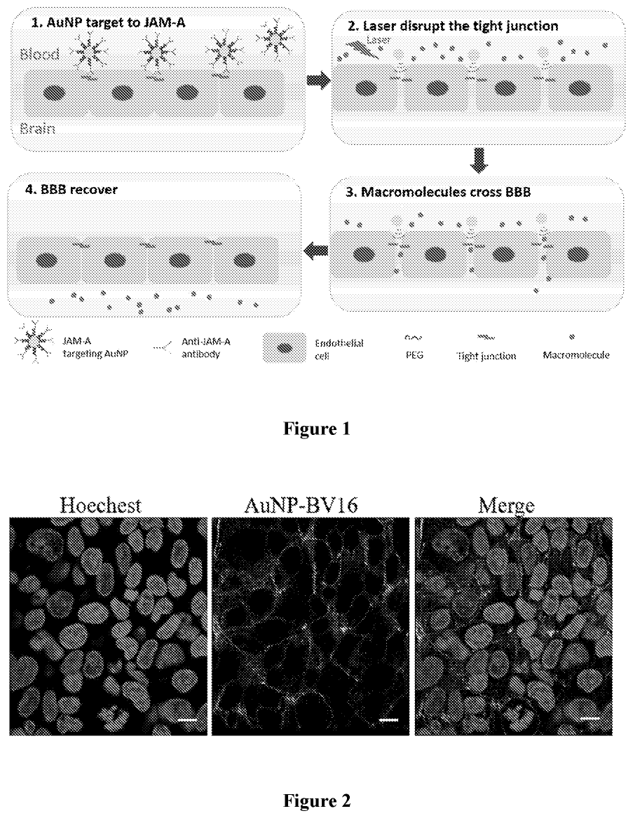

Here, the inventors designed and synthesized plasmonic AuNP to target the TJ protein, JAM-A, a critical component of the blood-brain barrier. By using short laser pulses such as picosecond and nanosecond pulses, they generated nanoscale heating and photomechanical effects to activate TJ-targeting AuNPs. As shown in schematic (FIG. 1), these excited nanoparticles locally disrupt the tight junction thus temporarily compromising the blood-brain barrier. The inventors tested and confirmed their hypothesis both in vitro using a cell monolayer model and in vivo.

This approach offers a new approach to brain drug delivery, such as for treating brain cancers. Specifically, for brain tumors such as glioblastoma, the optically triggered blood-brain barrier opening allows the use of more effective chemotherapy drugs from a large pool of anticancer drugs, previously unavailable due to their inability to pass through blood-brain barrier. With more effective therapies for hard to treat brain tumors, the inventors expect substantially reduced side effects that the brain cancer patients currently suffer from chemotherapy treatment, and as well improved survival and quality of life.

### I. BLOOD-BRAIN BARRIER

The blood-brain barrier (BBB) is a highly selective semipermeable border that separates the circulating blood from the brain and extracellular fluid in the central nervous system (CNS). The blood-brain barrier is formed by endothelial cells of the capillary wall, astrocyte end-feet ensheathing the capillary, and pericytes embedded in the capillary basement membrane. This system allows the passage of some molecules by passive diffusion, as well as the selective transport of molecules such as glucose, water and amino acids that are crucial to neural function. Specialized structures participating in sensory and secretory integration within neural circuits—the circumventricular organs and choroid plexus—do not have a blood-brain barrier. The blood-brain barrier restricts the passage of pathogens, the diffusion of solutes in the blood, and large or hydrophilic molecules into the cerebrospinal fluid (CSF), while allowing the diffusion of hydrophobic molecules (O₂, CO₂, hormones) and small polar molecules. Cells of the barrier actively transport metabolic products such as glucose across the barrier using specific transport proteins.

The blood-brain barrier results from the selectivity of the tight junctions between endothelial cells in CNS vessels, which restricts the passage of solutes. At the interface between blood and the brain, endothelial cells are stitched together by these tight junctions, which are composed of smaller subunits, frequently biochemical dimers, that are transmembrane proteins such as occludin, claudins, junctional adhesion molecule (JAM), or ESAM, for example. Each of these transmembrane proteins is anchored into the endothelial cells by another protein complex that includes ZO-1 and associated proteins.

The blood-brain barrier is composed of high-density cells restricting passage of substances from the bloodstream much more than do the endothelial cells in capillaries elsewhere in the body. Astrocyte cell projections called astrocytic feet (also known as “glia limitans”) surround the endothelial cells of the BBB, providing biochemical support to those cells. The BBB is distinct from the quite similar blood-cerebrospinal fluid barrier, which is a function of the choroidal cells of the choroid plexus, and from the blood-retinal barrier, which can be considered a part of the whole realm of such barriers.

Several areas of the human brain are not on the brain side of the BBB. Some examples of this include the circumventricular organs, the roof of the third and fourth ventricles, capillaries in the pineal gland on the roof of the diencephalon and the pineal gland. The pineal gland secretes the hormone melatonin “directly into the systemic circulation,” thus melatonin is not affected by the blood-brain barrier.

The blood-brain barrier acts effectively to protect the brain from circulating pathogens. Accordingly, blood-borne infections of the brain are rare. Infections of the brain that do occur are often difficult to treat. Antibodies are too large to cross the blood-brain barrier, and only certain antibiotics are able to pass. In some cases, a drug has to be administered directly into the cerebrospinal fluid (CSF) where it can enter the brain by crossing the blood-cerebrospinal fluid barrier.

The blood-brain barrier may become leaky in select neurological diseases, such as amyotrophic lateral sclerosis, epilepsy, brain trauma and edema, and in systemic diseases, such as liver failure. The blood-brain barrier becomes more permeable during inflammation, allowing antibiotics and phagocytes to move across the BBB. However, this also allows bacteria and viruses to infiltrate the blood-brain barrier. Examples of pathogens that can traverse the blood-brain barrier include Toxoplasma gondii which causes toxoplasmosis, spirochetes like Borrelia (Lyme disease), Group B streptococci which causes meningitis in newborns, and Treponema pallidum which causes syphilis. Some of these harmful bacteria gain access by releasing cytotoxins like pneumolysin which have a direct toxic effect on brain microvascular endothelium and tight junctions.

### II. BRAIN DISEASE

A. Cancer

Cancerous brain tumors can be divided into primary tumors, which start within the brain, and secondary tumors, which have spread from elsewhere, known as brain metastases. All brain tumors may produce symptoms that vary depending on the part of the brain involved. These symptoms may include headaches, seizures, visual field deficits, nausea and vomiting, language dysfunction, gait imbalance, and mental status changes. The headache is classically worse in the morning and goes away with vomiting. As the disease progresses, the level of consciousness will worsen.

The cause of most brain tumors is unknown. Uncommon risk factors include inherited neurofibromatosis, exposure to vinyl chloride, Epstein-Barr virus, and ionizing radiation. Studies on mobile phone exposure have not shown a clear risk. The most common types of primary tumors in adults are meningiomas (usually benign) and gliomas such as glioblastomas. In children, the most common type is medulloblastoma. Diagnosis is usually by neurologic examination along with computed tomography (CT) or magnetic resonance imaging (MRI) with contrast. The result is then often confirmed by a biopsy and or resection. Based on the results, the tumors are diagnosed by specific molecular mutational status and histologic subtype.

Treatment may include some combination of surgery, radiation therapy, and chemotherapy. Anticonvulsant medication may be needed if seizures occur. Dexamethasone may be used to decrease swelling around the brain tumor. Some tumors grow gradually, requiring only monitoring, and possibly needing no further intervention. Treatments that upregulates a person's immune system are being studied. Outcome varies considerably depending on the tumor type and its molecular characteristics. Glioblastomas usually have very poor outcomes, while benign meningiomas usually have good outcomes. The average five-year survival rate for all brain cancers in the United States is 33%.

Secondary, or metastatic, brain tumors are about five times more common than primary brain tumors, with about half of brain metastases coming from lung cancer. Primary brain tumors occur in around 250,000 people a year globally, making up less than 2% of cancers. In children younger than 15, brain tumors are second only to acute lymphoblastic leukemia as the most common form of cancer.

Anaplastic Astrocytoma. The histologic features of WHO grade III anaplastic astrocytomas are similar to those of low-grade astrocytomas, but there is more hypercellularity and nuclear and cellular pleomorphism. Cytoplasm may be scanty, with nuclear lobation and enlargement indicating anaplasia. Mitotic activity is easily recognized in most anaplastic astrocytomas but inexplicably may be absent in areas with gemistocytes.

The range of anaplasia in this grade is broad, with some examples showing low cellularity and pleomorphism with a few mitotic figures and others being highly cellular and pleomorphic with frequent mitoses, lacking only the pseudopallisading necrosis typical in the histologic diagnosis of glioblastoma. For this reason, it is useful to have a more objective indicator of behavior, such as markers of cellular proliferation. The most used markers have been antibodies to bromodeoxyuridine (BrdU) and Ki-67. The cellular incorporation of BrdU is a specific marker of the DNA synthesis phase of the cell cycle, whereas the Ki-67 antibody labels an antigen that is present in all phases of the cell cycle except Go. Both antibodies can be identified by immunohistochemical staining in paraffin-embedded tissue sections. As a generalization, higher labeling rates for anaplastic astrocytomas is associated with poor prognosis.

Glioblastoma multiforme. Glioblastoma multiforme (GBM) is the glioma with the highest grade of malignancy, WHO grade IV. It represents 15% to 23% of intracranial tumors and about 50%-60% of astrocytomas, and is the most common astrocytoma. They are considered to arise from astrocytes because glial fibrillary acidic protein can be identified in the cell cytoplasm. Autopsy and serial biopsy studies have shown that some astrocytomas transform from low-grade (WHO grade II) to anaplastic astrocytoma (WHO grade III) to glioblastoma (WHO grade IV). However, many GBMs arise de novo without passing through the lower grades of malignancy, and thus are diagnosed as Grade IV GBMs at initial presentation.

Pseudopallisading necrosis is the characteristic histologic feature that distinguishes GBM from anaplastic astrocytoma. Another microscopic feature that is distinctive and diagnostic is the presence of proliferative vascular changes within the tumor (e.g. microvascular proliferation). These changes may occur in the endothelial cells (vascular endothelial hyperplasia or proliferation) or in the cells of the vessel wall itself (vascular mural cell proliferation). Glioblastomas cellularity is usually extremely high. The individual cells may be small, with a high nuclear:cytoplasmic ratio, or very large and bizarre, with abundant eosinophilic cytoplasm. These same small cells may appear to condense in rows around areas of tumor necrosis, forming the characteristic pseudopallisades. GBMs have a propensity to infiltrate the brain extensively, spreading even to distant locations, thus giving the appearance of a multifocal glioma. Some examples are truly multifocal (i.e., arising in multiple simultaneous primary sites and possibly involving the contralateral cerebral hemisphere) while many of these multifocal tumors show a histologic connection when the whole brain is examined at autopsy.

Medulloblastoma. Medulloblastoma is the most common type of primary brain cancer in children. It typically originates in the cerebellum or posterior fossa. The brain is divided into two main parts, the larger cerebrum on top and the smaller cerebellum below towards the back of the skull. They are separated by a membrane called the tentorium. Tumors that originate in the cerebellum or the surrounding region below the tentorium are infratentorial in location. Historically medulloblastomas have been classified as primitive neuroectodermal tumors (PNET), but it is now known that medulloblastoma is distinct from supratentorial PNETs.

Medulloblastomas are invasive, rapidly growing tumors that, unlike most brain tumors, spread through the cerebrospinal fluid and frequently metastasize to different locations along the surface of the brain and spinal cord. Metastases involving the spinal cord are is termed “drop metastases.” The cumulative relative survival rate for all age groups and histology follow-up was 60%, 52%, and 47% at 5 years, 10 years, and 20 years, respectively, with children doing better than adults.

Signs and symptoms are mainly due to secondary increased intracranial pressure due to blockage of the fourth ventricle; tumors are usually present for 1 to 5 months before diagnosis is made. The child typically becomes listless, with repeated episodes of vomiting, and morning headaches which may lead to a misdiagnosis of gastrointestinal disease or migraine. Soon after, the child can develop a stumbling gait, truncal ataxia, frequent falls, diplopia, papilledema, and sixth cranial nerve palsy. Positional dizziness and nystagmus are also frequent, and facial sensory loss or motor weakness may be present.

Extraneural metastasis to the rest of the body is rare, and when it occurs, it is in the setting of relapse, more commonly in the era prior to routine chemotherapy. Medulloblastomas are usually found in the vicinity of the fourth ventricle. Tumors with similar appearance and characteristics originate in other parts of the brain, but they are not identical to medulloblastoma.

Although medulloblastomas are thought to originate from immature or embryonal cells at their earliest stage of development, the cell of origin depends on the subgroup of medulloblastoma. WNT tumors originate from the lower rhombic lip of the brainstem, while SHH tumors originate from the external granular layer.

Currently, medulloblastomas are thought to arise from cerebellar stem cells that have been prevented from dividing and differentiating into their normal cell types. This accounts for the histologic variants seen on biopsy. Both perivascular pseudorosette and Homer Wright rosette pseudorosette formation are highly characteristic of medulloblastomas, and are seen in up to half of medulloblastomas.

Oligodendrogliomas. Like astrocytomas, oligodendrogliomas mimic the histology of their presumed cell of origin. They also arise primarily in the white matter but tend to infiltrate the cerebral cortex more than astrocytomas of a similar grade. Like astrocytomas, grading schemes of histologic malignancy have been used for oligodendrogliomas, but these correlate less well with prognosis than those used for astrocytomas. Many of the histologic features used to grade oligodendrogliomas are similar to those used for astrocytomas, including cellularity, pleomorphism, mitotic activity, vascular changes, and necrosis. Oligodendrogliomas of all histologic grades tend to infiltrate the cortex readily and to form clusters of neoplastic cells in the subpial region, around neurons, and around blood vessels. In general, oligodendrocytes have round regular nuclei and distinct cytoplasmic borders with clearing of the cytoplasm. Another fairly distinctive histologic feature is the vascular pattern of oligodendrogliomas, referred to as “chicken-wire” vessels that can divide the tumor into discrete lobules. The pathognomonic molecular feature of oligodendrogliomas is the codeletion of chromosomes 1p and 19q, which is associated with increased responses to chemotherapy and radiation therapy as well as improved survival outcomes compared to astrocytomas, which lack the codeletion of chromosomes 1p and 19q. Anaplastic oliogdendrogliomas have notably better prognoses compared to anaplastic astrocytomas and GBMs. Some authors have reported that a MIB-1 labeling index of >3%-5% predicts a worse prognosis in oligodendrogliomas.

B. Infections

A wide variety of infections of the brain are known. These can be generally categorized as fungal, protozoal, bacterial, viral and prionic. There are also a number of post-infection syndromes affecting the central nervous system. Examples are set out below:

Fungal:
Cryptococcal meningitis
Brain abscess
Spinal epidural infection

Protozoal:
Toxoplasmosis
Malaria
Primary amoebic meningoencephalitis

Bacterial:
Tuberculosis
Leprosy
Neurosyphilis
Bacterial meningitis
Late stage Lyme disease
Brain abscess
Neuroborreliosis

Viral:
Viral meningitis
Eastern equine encephalitis
St Louis encephalitis
Japanese encephalitis
West Nile encephalitis
Tick-borne encephalitis
Herpes simplex encephalitis
Rabies
California encephalitis virus
Varicella-zoster encephalitis
La Crosse encephalitis
Measles encephalitis
Nipah virus encephalitis
Poliomyelitis
Slow virus infections, which include:
   Subacute sclerosing panencephalitis
   Progressive multifocal leukoencephalopathy
   Acquired immunodeficiency syndrome (AIDS)

Prionic:
Creutzfeldt-Jakob disease
Fatal familial insomnia
Gerstmann-Sträussler-Scheinker syndrome
Kuru

Post-Infectious Diseases of the Central Nervous System:
PANDAS
Sydenham's chorea
Acute disseminated encephalomyelitis
Guillain-Barré syndrome

C. Other Brain Disorders

A host of other brain disorders exist for which improved delivery of a therapeutic to the brain could greatly enhance treatment options and positive patient outcomes. Examples in autonomic nervous system disorders such as dysautonomia and Multiple System Atrophy, seizure disorders such as epilepsy, movement disorders of the central and peripheral nervous system such as Parkinson's disease, Essential tremor, Amyotrophic lateral sclerosis, Tourette's Syndrome, Multiple Sclerosis and various types of Peripheral Neuropathy, sleep disorders such as Narcolepsy, migraines and other types of Headache such as Cluster Headache and Tension Headache, neuropsychiatric illnesses (diseases and/or disorders with psychiatric features associated with known nervous system injury, underdevelopment, biochemical, anatomical, or electrical malfunction, and/or disease pathology, e.g., Attention deficit hyperactivity disorder, Autism, Tourette's syndrome and some cases of obsessive compulsive disorder as well as the neurobehavioral associated symptoms of degeneratives of the nervous system such as Parkinson's disease, essential tremor, Huntington's disease, Alzheimer's disease, multiple sclerosis and organic psychosis), delirium and dementias such as Alzheimer's disease, dizziness and vertigo, and stroke (CVA, cerebrovascular attack).

### III. PLASMONIC NANOPARTICLES

Plasmonic nanoparticles are particles whose electron density can couple with electromagnetic radiation of wavelengths that are far larger than the particle due to the nature of the dielectric-metal interface between the medium and the particles: unlike in a pure metal where there is a maximum limit on what size wavelength can be effectively coupled based on the material size. Common materials for plasmonic nanoparticles are noble metals, such as gold and silver.

What differentiates these particles from normal surface plasmons is that plasmonic nanoparticles also exhibit interesting scattering, absorbance, and coupling properties based on their geometries and relative positions. These unique properties have made them a focus of research in many applications including solar cells, spectroscopy, signal enhancement for imaging, and cancer treatment. As well owing to their high sensitivity appear to be good candidates for designing mechano-optical instrumentation.

Plasmons are the oscillations of free electrons that are the consequence of the formation of a dipole in the material due to electromagnetic waves. The electrons migrate in the material to restore its initial state; however, the light waves oscillate, leading to a constant shift in the dipole that forces the electrons to oscillate at the same frequency as the light. This coupling only occurs when the frequency of the light is equal to or less than the plasma frequency and is greatest at the plasma frequency that is therefore called the resonant frequency. The scattering and absorbance cross-sections describe the intensity of a given frequency to be scattered or absorbed. Many fabrication processes or chemical synthesis methods exist for preparation of such nanoparticles, depending on the desired size and geometry.

The nanoparticles can form clusters (the so-called “plasmonic molecules”) and interact with each other to form cluster states. The symmetry of the nanoparticles and the distribution of the electrons within them can affect a type of bonding or antibonding character between the nanoparticles similarly to molecular orbitals. Since light couples with the electrons, polarized light can be used to control the distribution of the electrons and alter the mulliken term symbol for the irreducible representation. Changing the geometry of the nanoparticles can be used to manipulate the optical activity and properties of the system, but so can the polarized light by lowering the symmetry of the conductive electrons inside the particles and changing the dipole moment of the cluster. These clusters can be used to manipulate light on the nano scale.

In one example, gold nanoparticle (AuNP) can be prepared with different sizes (15, 30, 40 and 60 nm). The citrate-capped AuNP functionalized with OPSS-PEG-SVA is conjugated with an anti-JAM-A antibody (BV16) and backfilled with polyethylene glycol (PEG) at a density of 6 mPEG/nm² to increase the specificity of the targeting. The AuNP are functionalized with BV16 to target hCMEC/D3 cells expressing JAM-A, one of the tight junction molecules. Upon antibody coating, the particle size increases by 18-34 nm as determined by dynamic light scattering (DLS). TJ-targeting by AuNP can be confirmed by UV-vis spectroscopy, which show the absorbance peak shift of functionalized AuNP of approximately 2 nm.

Other nanoparticles, such as magnetic nanoparticles and CuS particles having a broad absorption of light, are contemplated.

### IV. METHODS OF DELIVERING AGENTS TO THE BRAIN

In accordance with the present disclosure, the inventors propose the use of ultrashort light pulses to excite light absorbing particles and disrupt tight junction leading to temporary permeability increase of the blood-brain barrier. A highly useful embodiment involves the use of ultrashort pulse lasers.

An ultrashort pulse laser is a laser that emits ultrashort pulses of light, generally of the order of femtoseconds to ten nanoseconds or longer. They are also known as ultrafast lasers owing to the speed at which pulses turn “on” and “off” (not to be confused with the speed at which light propagates, which is determined by the properties of the medium, and has an upper limit), particularly its index of refraction, and can vary as a function of field intensity (i.e., self-phase modulation) and wavelength (chromatic dispersion).

Lasers can be delivered to the brain in free space during surgery or with a transparent skull implant. Alternatively, lasers can be delivered to the brain by inserting or implanting an optical fiber into defined brain regions.

Common current ultrashort pulse laser technologies include Ti-sapphire lasers, Nd:YAG lasers, dye lasers. High output peak power usually requires chirped pulse amplification of a seed pulse from a mode-locked laser. Dealing with high optical powers also needs the nonlinear optical phenomena to be taken in account.

### V. PHARMACEUTICAL FORMULATIONS

The delivery of agents in accordance with the present disclosure requires preparation of pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render agents stable and allow for uptake by the brain. Aqueous compositions of the present disclosure thus comprise an effective amount of the agent dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the agents of the compositions.

The agents of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection. Such compositions would normally be administered as pharmaceutically acceptable compositions, as described supra.

By way of illustration, solutions of the agents as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The following is an exemplary and non-limiting disclosure of agents for use in accordance with the presently disclosed compositions and methods.

A. Common Agents for the Treatment of Brain Cancer

PCV is a drug combination therapy employing three different agents—a hydrazine derivative, matulane, a nitrosourea, lomustine, and a tubulin interactive agent, vincristine. It has been used in a number of clinical trials, most notably by the inventor in assessing its effect on high-grade glioma and medulloblastoma tumors. The major side-effect observed with PCV was dose-limiting myelotoxicity. Each of the components of PCV is described below. It should be noted that one could use BCNU rather than of CCNU (lomustine) since both are nitrosoureas. It also is contemplated that one could use CCNU and procarbazine or BCNU and procarbazine, without vincristine, since vincristine is usually considered to be the least active of the drugs in the PCV combination.

In addition to hydrazine and nitrosoureas, alkylating agents include triazenes such as dacarabzine and temozolomide, nitrogen mustards such as chlorambucil, cyclophosphamide, isofamide, mechlorethamine, melphalan, uracil mustard; aziridine such as thiotepa; methanesulphonate esters such as busulfan; platinum complexes such as cisplatin, carboplatin; bioreductive alkylators, such as mitomycin and altretemine. Any of these compounds may be used together or individually, in combination with the compounds of the present disclosure.

Hydrazine and triazene derivatives are similar to nitrosoureas in that they decompose spontaneously or are metabolized to produce alkyl carbonium ions, which alkylate DNA. This class of compounds includes matulane, dacarbazine and temozolomide.

The active ingredient in matulane is Procarbazine Hydrochloride (N-isopropl-alpha-(2-methylhydrazino)-p-toluamide monohydrochloride). It is available from Roche Laboratories, Inc. It was approved in 1969 for treatment of Hodgkins' Disease. The typical form is an oral capsule that contains 50 mg procarbazine as the hydrochloride. Dosages vary depending upon whether procarbazine is being used as a combination drug with other anticancer drugs or as a single therapeutic agent. A suggested guideline per the PDR for single agent use is 100 mg two times daily for 14 days.

Nitrosoureas represent a group of therapeutic alklyating agents. This class of compounds includes lomustine, carmustine, semustine, steptozocin, and nimustine.

Lomustine is a synthetic alkylating agent, also known as CCNU, with the chemical name of 1-(2-chloroethyl)-3-cyclohexyl-1-nitrosourea. It was approved in 1977 for treatment of brain tumors and Hodgkin's Disease. It is available from Bristol Myers Squibb as oral capsule, available in 10 mg, 40 mg and 100 mg forms. Dosages may vary depending upon whether lomustine is being used as a single agent or in a combination in addition to other chemotherapeutic agents. As a single agent in previously untreated patients, the recommended dosages per the PDR is 130 mg as a single oral dose every 6 weeks. Lomustine crosses the blood-brain barrier.

Carmustine, also known as BCNU, with the chemical name of N,N′-Bis(2-chloroethyl)-N-nitrosurea, is a nitrosurea alkylating agent approved by the FDA in 1977. Carmustine has been used for many years for treatment of primary brain tumors and is used for the treatment of gliomas. Carmustine is available from Bristol Meyers Squibb in packages containing vials of 10 mg carmustine and 3 ml sterile diluent for delivered by i.v. injection. As a single agent carmustine is administered at about 150-200 mg/m² every 6 weeks. In combination regimens, carmustine may be given in does similar to those of lomustine. An alternative mode of delivery is by wafers implanted directly into the tumor site (Gliadel® Wafer).

Tubulin interactive agents interfere with cell division by binding to specific sites on Tubulin, a protein that polymerizes to form cellular microtubules. Microtubules are critical cell structure units. When the interactive agents bind on the protein, the cell cannot properly form microtubules. Tubulin interactive agents include vincristine and vinblastine, both alkaloids and the taxanes, such as paclitaxel and docetaxel.

Vincristine is available as Oncovin™ from Eli Lilly & Company and as Vincristine Sulfate from Faulding. Also called vincaleukoblastine, a 22-oxo-, sulfate (1:1) (salt), the salt of an alkaloid obtained from a common flowering herb, the periwinkle plant. It is delivered by intravenous injection. It was approved in 1963 on label for Ewing's Sarcoma, rhabdomyosarcoma, Wilm's Tumor, neuroblastoma, Hodgkin's Disease and leukemia.

Numerous highly proliferative types of cancer, including brain cancers, are associated with increased levels of the polyamines putrescine, spermidine, and spermine in tumor tissue and blood and urine of mammals with cancer. Studies have shown that this can be related to increased polyamine synthesis by the rate-limiting enzyme, ornithine decarboxylase (ODC). The pathway for polyamine synthesis begins with L-ornithine. DFMO (α-difluoromethylornithine, eflornithine, Ornidyl®) is a structural analog of the amino acid L-ornithine and has a chemical formula C₆H₁₂N₂O₂F₂. DFMO can be employed in the methods of the disclosure as a racemic (50/50) mixture of D- and L-enantiomers, or as a mixture of D- and L-isomers where the D-isomer is enriched relative to the L-isomer, for example, 70%, 80%, 90% or more by weight of the D-isomer relative to the L-isomer. The DFMO employed may also be substantially free of the L-enantiomer.

Other drugs approved for treating brain cancers include everolimus, bevacizumab and temozolomide. Everolimus (marketed as Afinitor) is the 40-O-(2-hydroxyethyl) derivative of sirolimus and works similarly to sirolimus as an inhibitor of mammalian target of rapamycin (mTOR). It is currently used as an immunosuppressant to prevent rejection of organ transplants and in the treatment of renal cell cancer and other tumours. Much research has also been conducted on everolimus and other mTOR inhibitors as targeted therapy for use in a number of cancers.

Bevacizumab (tradename Avastin) is a medication used to treat a number of types of cancers and a specific eye disease. For cancer it is given by slow injection into a vein and used for colon cancer, lung cancer, glioblastoma, and renal-cell carcinoma. For age-related macular degeneration it is given by injection into the eye. Common side effects when used for cancer include nose bleeds, headache, high blood pressure, and rash. Other severe side effects include gastrointestinal perforation, bleeding, allergic reactions, blood clots, and an increased risk of infection. When used for eye disease side effects can include vision loss and retinal detachment. Bevacizumab is in the angiogenesis inhibitor and monoclonal antibody families of medication. It works by slowing the growth of new blood vessels by inhibiting vascular endothelial growth factor A (VEGF-A).

Temozolomide (TMZ; brand names Temodar and Temodal and Temcad) is an oral chemotherapy drug. It is an alkylating agent used as a treatment of some brain cancers, as a second-line treatment for astrocytoma and a first-line treatment for glioblastoma multiforme.

B. Antibiotics/Antivirals/Antifungals

1. Antibiotics

The term “antibiotics” are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

The first commercially available antibiotic was released in the 1930's. Since then, many different antibiotics have been developed and widely prescribed. In 2010, on average, 4 in 5 Americans are prescribed antibiotics annually. Given the prevalence of anitbiotics, bacteria have started to develop resistance to specific antibiotics and antibiotic mechanisms. Without being bound by theory, the use of antibiotics in combination with another antibiotic may modulate resistance and enhance the efficacy of one or both agents.

In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

2. Antivirals

The term “antiviral” or “antiviral agents” are drugs which may be used to treat a viral infection. In general, antiviral agents act via two major mechanisms: preventing viral entry into the cell and inhibiting viral synthesis. Without being bound by theory, viral replication can be inhibited by using agents that mimic either the virus-associated proteins and thus block the cellular receptors or using agents that mimic the cellular receptors and thus block the virus-associated proteins. Furthermore, agents which cause an uncoating of the virus can also be used as antiviral agents.

The second mechanism of viral inhibition is preventing or interrupting viral synthesis. Such drugs can target different proteins associated with the replication of viral DNA including reverse transcriptase, integrase, transcription factors, or ribozymes. Additionally, the therapeutic agent interrupts translation by acting as an antisense DNA strain, inhibiting the formation of protein processing or assembly, or acting as virus protease inhibitors. Finally, an anti-viral agent could additionally inhibit the release of the virus after viral production in the cell.

Additionally, anti-viral agents could modulate the body's own immune system to fight a viral infection. Without being bound by theory, the anti-viral agent which stimulates the immune system may be used with a wide variety of viral infections.

In some embodiments, the present disclosure provides methods of using the disclosed compounds in a combination therapy with an anti-viral agent as described above. In some non-limiting examples, the anti-viral agent is abacavir, aciclovir, acyclovir, adefovir, amantadine, amprenavir, ampligen, arbidol, atazanavir, atripla, balavir, boceprevirertet, cidofovir, combivir, dolutegravir, daruavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, interferon type I, type II, and type III, lamivudine, lopinavir, loviride, maraviroc, moroxydine, methisazone, nelfinavir, nevirapine, nexavir, oseltamivir, penciclovir, peramivir, pleconaril, podophyllotoxin, raltegravir, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, traporved, valaciclovir, valganciclovir, vicriviroc, vidarabine, viramidine, zalcitabine, zanamivir, or zidovudine. In some embodiments, the anti-viral agents is an anti-retroviral, a fusion inhibitor, an integrase inhibitor, an interferon, a nucleoside analogues, a protease inhibitor, a reverse transcriptase inhibitor, a synergistic enhancer, or a natural product such as tea tree oil.

3. Antifungals

Antifungal agents can be grouped in the following categories allylamines, azoles (imidazoles, triazoles), polyenes, and echinocandins. Allylamines include amorolfine, butenafine, naftifine and terbinafine. Azoles include bifonazole, butoconazole, clotrimazole, econazole, fenticonazole, ketoconazole, isoconazole, luliconazole, miconazole, omoconazole, oxiconazole, sertaconazole, sulconazole, tioconazole, and terconazole. Triazoles include albaconazole, efinaconazole, fluconazole, isavuconazole, posaconazole, ravuconazole, terconazole, and voriconazole. Abafungin is a thiazole. Polyenes include amphotericin B, natamycin and trichomycin. Echinocandins include anidulafungin, caspofungin and micafungin.

Other miscellaneous antifungals include arylguanidines, tolnaftate, flucytosine, butenafine, griseofulvin, ciclopirox, selenium sulfide and tavaborole.

### VI. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute specifically contemplated modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Materials. Human cerebral microvessel endothelial cell/D3 cell line, EndoGRO™-MV Complete Media Kit, FGF-2, trypsin-EDTA, collagen type I, were purchased from Millipore. 532 nm picosecond (ps) was purchased from EKSPLA. Antibody BV16 and BV11 were from Prof. Dejana Lab (IFOM, Milan, Italy). Gold (III) chloride, FITC-dextran, DMSO, hydroquinone, sodium citrate tribasic, BSA, Tween 20, triton-X 100, sodium carbonate, sodium bicarbonate, and sucrose were purchase from Sigma-Aldrich. Pen-strep, donkey anti-mouse Ig G (H+L) were purchase from Life Technologies. OPSS-PEG-SVA, mPEG-Sh were purchased from Laysan Bio, Inc. Donkey serum, goat serum, Trypan blue, gold reference standard solution, Hoechst 33342, DAPI, MTT assay kit, Dulbecco's phosphate buffered saline, 20 kDa dialysis membrane, 6-, 24-, 96-well plates, and cell culture insert were purchased from Thermo Fisher Scientific. All other chemicals were analytical grade. Adult mice were ordered from Charles River Laboratories. Animal protocols were approved by Institutional Animal Care Use Committee (IACUC) of University of Texas at Dallas.

Gold nanoparticle synthesis. Plasmonic gold nanoparticle (AuNP) preparation involved the chemical reduction of gold chloride using sodium citrate to get the size of 15-200 nm AuNP by previous report^{11,12}. Specifically, to make the 15 nm AuNP, 98 ml of ultrapure (UP) water and 1 ml 25 mM gold chloride are mixed in the clean glass flask on a hot plate with 300° C. 1 ml of 112.2 mM sodium citrate is quickly added to the boiling and stirring mixture. With heat and stir, the color of the solution is changed from purple to red gradually in 10 minutes. The AuNP solution is taken from hot plate when the reaction is complete. We performe dynamic light scattering (DLS) and UV-spectroscopy to characterize the AuNP.

To make large AuNP, 15 mM sodium citrate tribase and 25 mM hydroquinone solution are prepared. The appropriate amount of UP water is added to a clean glass flask based on table 1. Under vigorous stirring, the remaining reagents from table 1 are added in the following order: gold chlroride solution, sodium citrate and hydroquinone. The color of solution change from purple to red immediately after the last component hydroquinone is added into the vortex.

TABLE 1
Synthesis of AuNP with size 30-200 nm

| | | 2.5*10⁻²M | 1.5*10⁻²M | 2.23 nM | 2.5*10⁻²M |
|---|---|---|---|---|---|
| Size | H₂O | HAuCl₄ | sodium | 15 nm | hydroquinone |
| (nm) | (ml) | (ml) | citrate (ml) | seeds (ml) | (ml) |
| 30 | 85.63 | 0.8750 | 0.8750 | 12.5 | 0.8750 |
| 45 | 94.33 | 0.9629 | 0.9629 | 3.71 | 0.9629 |
| 60 | 96.45 | 0.9843 | 0.9843 | 1.57 | 0.9843 |
| 80 | 97.35 | 0.9934 | 0.9934 | 0.66 | 0.9934 |
| 100 | 97.66 | 0.9966 | 0.9966 | 0.34 | 0.9966 |
| 150 | 97.90 | 0.9990 | 0.9990 | 0.1 | 0.9990 |
| 200 | 97.96 | 0.9996 | 0.9996 | 0.0422 | 0.9996 |

Cell culture. Human cerebral microvessel endothelial cell/D3 (hCMEC/D3) cells were cultured in EndoGRO™-MV Complete Media supplemented with FGF-2 on a collagen-coated porous membrane. Once plated to the 24 well plate transwell filter insert, the D3 cells were cultivated approximately 6 to 7 days to form cellular monolayer at 37° C. and 5% CO₂. Extensive characterization of hCMEC/D3 monolayer was performed by transendothelial electrical resistance (TEER), permeability, and immunocytochemistry staining.

Transendothelial electrical resistance (TEER) measurement. The electrical resistance of hCMEC/D3 monolayers were measured in Ohms (Ω). A cellular monolayer was seeded on transwell inserts (0.3 cm², pore size 8 μm) that include upper and bottom compartments. For TEER measurement, cellular monolayers were detected by epithelial voltmeter (Millicell ERS-2, Millipore, USA). Rinsing of electrodes by cell culture media was required between blank well and sample-well. The TEER values of cellular monolayer were measured before (−0.5 hours) and at various time delays after laser irradiation (0, 0.5, 1, 1.5, 3 and 6 hours). The resistance value of a blank culture insert coated with collagen on the top side of the membrane was used as baseline, and the inventors subtract this baseline value from the resistance measured from the cell monolayer samples to obtain TEER. The resulting resistance value multiplied by effective membrane area gives the TEER value in Ω·cm².

Permeability measurement. All the media of cellular monolayers were replaced by cell culture media without 5% FBS from the top and chambers, followed by a half hour incubation in 37° C. and 5% CO² incubator. After that, 0.3 ml 1 mg/ml FITC-dextran mixed with media was added to the upper compartments. At selective time points, 100 μl media were aspirated from the bottom well and added into 96 black well plates. 100 μl of culture media was then replaced in the bottom chamber to keep constant volume. The inventors then measured the fluorescent intensity for the collected samples in the 96-well plate (excitation at 490 nm and emission at 540 nm) to obtain FITC-dextran concentration. The quantity (Q) was obtained by concentration multiplying cleared volume. The apparent permeability (Papp, cm/sec) was calculated by rate of FITC-dextran quantity change over time (dQ/dt), divided by the initial concentration of dextran (C) and the membrane area (A).

$${P}_{app} = \frac{dQ}{dt} * \frac{1}{A * C}$$

Immunocytochemistry staining. The JAM-A expression on hCMEC/D3 cells was detected by ICC staining. Cellular monolayers were fixed for 5 minutes in methanol on ice, then washed in PBS 3 times. A blocking buffer (5% donkey serum 2% BSA in PBS 0.05% Tween) was applied at room temperature (RT) for 1 hour. Then the cells were incubated with the primary antibody (3-5 μg/ml BV16 in 5% donkey serum 2% BSA in PBS 0.05% Tween 20) overnight at 4° C. or RT for 1 hour, followed by a second antibody incubation for 1 hour at RT. Finally, the samples were incubated with Hoechst dye for 7 minutes in dark. The samples were washed in PBS every time before changing reagents. FV3000RS confocal microscopy was used to take images.

Gold nanoparticle conjugation with antibodies. AuNPs were synthesized by previous reported method¹¹. Then AuNP surface were modified by antibodies to target TJ protein, JAM-A. Specifically, BV16 or BV11, an anti-JAM-A antibody, were diluted to 0.5 mg/ml in PBS, followed by dilution in aqueous 2 mM borate buffer at pH=8.5. OPSS-PEG-NHS was dissolved in borate buffer and quickly added to the diluted antibody at 125:1 molar ratio. The mixture was vortexed briefly and kept for 3 hours shaking on ice, followed by dialysis for 3 hours to remove free OPSS-PEG-NHS through 20 kDa MW membrane. The thiolated BV16 or BV11 was reacted with concentrated AuNPs at molar ratio of 200:1 at RT for 1 hour on ice. To stabilize AuNP-BV16, polyethylene glycol (PEG) were added at 6 PEG/nm³ for backfilling¹³ the empty space of AuNPs for 1 hour on ice. Finally, the modified AuNPs were washed 3 times and characterized by dynamic light scattering (DLS), UV-vis spectroscopy and TEM. Co-localization of AuNP modified by anti-JAM-A antibody and TJ were performed by immunocytochemistry (ICC) staining to confirm AuNP targeting.

Optical opening of BBB in vitro. To investigate the BBB temporary opening induced by 532 nm picosecond (ps) laser excite TJ-targeting AuNPs in vitro, hCMEC/D3 cells were seeded on transwell inserts for about 1 week to form monolayer with TEER value of around 60 Ω·cm², Pₐₚₚ of 8.38±1.17×10⁻⁷ cm/s for 40 kDa FITC-dextran. Expression of JAM-A in D3 cell was confirmed by ICC staining. The culture was incubated with 0.5 nM AuNP-BV16 for 0.5 hour at 37° C. and 5% CO₂. The treated cellular monolayer was washed three times with cell culture media before a ps laser was applied to the cellular monolayer. The BBB opening in vitro were characterized by TEER and permeability measurement.

MTT assay. The hCMEC/D3 cells were seeded on 96-well plates for about 1 week to form monolayer. The media were replaced by 0.5 mg/ml MTT reagent in cell culture media, followed by 4 hours incubation at 37° C. and 5% CO₂. After incubation, MTT reagent were replaced by 200 μl DMSO. The 96-well plates were wrapped up in foil and shaken for 20 minutes at RT. Finally, the plates were read at 590 nm.

Transcranial optical of BBB opening in vivo. C57BL/6 mice (7 weeks old, 22-24 g) were used for the in vivo experiments. The animal was anesthetized by 2-3% isoflurane and intravenously administrated 36.9 μg/g AuNPs functionalized by JAM-A antibody, BV11. Evans blue (2% in PBS) was injected from the tail vein at 100 μl, followed by a ps laser application. Specifically, 1% lidocaine mixed with 0.5% bupivacaine were subcutaneous injected on the brain locally. Then, the scalp was peeled back to expose the skull. Finally, the ps laser was applied through the skull to excite the AuNP in blood vessels. After 0.5 hr, mice were transcardially perfused with 30 ml PBS to remove the intravascular dye, followed by 4% PFA. The brains and other organs were extracted for Evans blue extravasation imaging and histology staining.

To measure the penetration of functional molecule, the anti-cancer drug, doxorubicin, and antibody (human IgG) instead of Evans blue dye were inject intravenously. To test endogenous mouse IgG extravasation, the secondary antibody was incubated with mouse brain tissue sections, followed by fluorescent signal detection.

Monte Carlo simulation of light penetration in mouse brain tissue. Monte Carlo simulation of photon propagation in tissue is conducted by MCML program developed by

Wang et al. (Computer Methods and Programs in Biomedicine 47(2) 131-146, 1995). The optical properties of different tissues (Table 2) used in this paper were extracted from previous experiment report¹⁴⁻¹⁷. The inventors first calculated the light fluence profile with an infinite light point source by MCML. Then, to obtain the finite beam light propagation profiles, the point source results were used for convolution over a Gaussian beam profile with Matlab (2016a) (convolution for responses to a finite diameter photon beam incident on multi-layered tissues). Finally, the inventors calculated beam scanning light penetration profile by composition of the single pulse profile.

The geometry of brain in the simulation was built according to the mouse brain atlas (Franklin et al., 2007). There are five layers in the model. The first layer is skull with thickness of 0.03 cm. The meninges was laid in between of skull and brain with a thickness of 0.01 cm. Because mouse meninges is very thin and contains mostly liquid, the inventors use CSF properties for this layer in the simulation. The grey matter with thickness of 0.1 cm corresponding to the cortex, following a layer of white matter with thickness of 0.03 cm. And the bottom layer is grey matter again with thickness greater than 0.4 cm. The laser beam profile can be described by Gaussian pulse formula:

$$G ( r ) = \frac{2 P}{π {R}^{2}} exp ( - 2 \frac{{r}^{2}}{{R}^{2}} )$$

where R is the 1/e² diameter of 0.41 cm (FWHM=0.29 cm), which was obtained from blade edge experiment measurements. Briefly, a blade was used to cover the beam gradually and pulse energy was measured simultaneously. By fitting the data with Gaussian beam model, the beam size was obtained (Khosrofian, J. M., Garetz, B. A. Applied Optics, Vol. 22, 1983, 21, 3406-3410]. P is the total energy of single laser pulse irradiation (0.03 mJ). The results are analyzed by Matlab 2016a (results shown below):

TABLE 2
The optical property of different mouse brain tissue

| | | | | | | μₛ′ = μₛ(1 − g)[cm⁻¹]ⁿ | | |
|---|---|---|---|---|---|---|---|---|
| | | | μₐ [cm⁻¹] | μₛ [cm⁻¹] | g | Reduced | | |
| | | d [cm] | Absorption | Scattering | Anisotropy | scattering | Refractive | |
| λ = 532 nm | | Thickness | coefficient | coefficient | factor | coefficient | index | Ref |
| Skull | | 3.00E-02 | 13.6 | 351.4 | 0.93 | 24.6 | 1.5 | [14] |
| bone | | | | | | | | |
| Meninges CSF | | 1.00E−02 | 0.004 | 2.5 | 0.001 | 2.5 | 1.33 | [16] |
| | | | | | | | | [17] |
| Cerebral | grey | 1.00E−01 | 0.44 | 100 | 0.88 | 12 | 1.3951 | [15] |
| cortex | matter | | | | | | | |
| | white | 3.00E−02 | 0.95 | 423 | 0.806 | 82.1 | 1.4121 | [15] |
| | matter | | | | | | | |

Immunohistochemistry staining. The mouse brains were dehydrated in 20% sucrose solution before cryosection processing. The mouse brain was processed with 20 μm thickness on a freezing cryostat. The sections were washed in PBS 3 times to remove cryoprotectant solution completely before blocking solution was applied. Free-floating sections were incubated with relevant parimary antibodies for 48 hours at 4° C. after blocking by 5% normal donkey serum with 0.1% Triton X-100 for 1 hour at RT. Secondary antibodies then were added, followed by incubation with DAPI solution. Finally, treated sections were mounted to glass slides carefully and imaged by confocal microscopy. Washing by PBS is necessary when changing solution for the staining procedure.

Biodistribution study. To quantitatively measure AuNP distribution in vivo, the inventors perfused the animal and collected the major at 1 hour, 6 hours, 12 hours and 24 hours after intravenous injection of AuNPs (n=3). The tissues were then digested in aqua regia and centrifuged at 5 000 g for 5 minutes. The gold concentration was analyzed by ICP-MS system to test gold concentration (% injection dose/g, or % ID/g).

To microscopically examine AuNP-BV11 distribution, the major organs were excised for histology staining using silver enhancement regents. The paraffin embedded brain tissue with 20 μm was processed in HistoPathology core at UT Southwestern Medical Center, followed by deparaffined step. The sections, then, were rinsed by water and incubated with Li silver enhancement developer for 20 minutes at room temperature. The reaction was stopped by water, followed by counterstaining by diluted hematoxylin. Finally, the inventors mounted the sections with synthetic mounting media for microscopy observation.

In vivo toxicity measurement of TJ-targeting AuNP. To investigate the dose safety of TJ-targeting AuNP (AuNP-BV11), the animals were weighed to check any body weight loss (BWL) post 36.9 μg/g nanoparticle injection and saline as control group. The inventors further observed any pathological changes in the histology analysis of major organs by comparing the nanoparticle injection versus control animal group.

### Example 2—Results

In vitro BBB model characterization. Human cerebral microvessel endothelial cell/D3 (hCMEC/D3) cells were used as in vitro BBB model. First, the inventors performed extensive characterization of hCMEC/D3 monolayer by transendothelial electrical resistance (TEER, 60 Ω·cm²), permeability (apparent permeability coefficient Pₐₚₚ=8.38±1.17×10⁻⁷ cm/s for 40 kDa FITC-dextran), and immunocytochemistry staining (JAM-A, FIG. 27). These values are in agreement with literature reported values¹⁸.

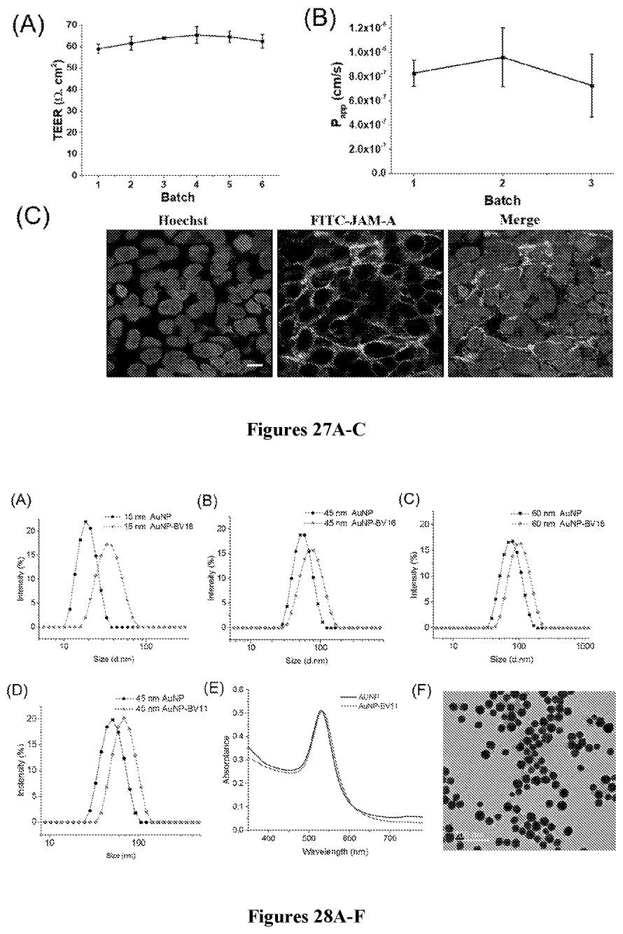

Synthesis of the tight junction (TJ)-targeting gold nanoparticles. The inventors synthesized AuNP with different sizes (15, 45 and 60 nm) conjugated with anti-JAM-A antibodies (AuNP-BV16 targeting hCMEC/D3 cell, and AuNP-BV11 for mice), while backfilling with polyethylene glycol (PEG) at a density of 6 mPEG/nm² to increase the specificity of the targeting¹³. Upon antibody coating, the particle size increased by 18-34 nm as determined by dynamic light scattering (DLS) (FIGS. 28A and 28B) and the absorbance peak shifted by 2-4 nm (FIG. 28C). TEM imaging confirms the size of AuNP (FIG. 28D).

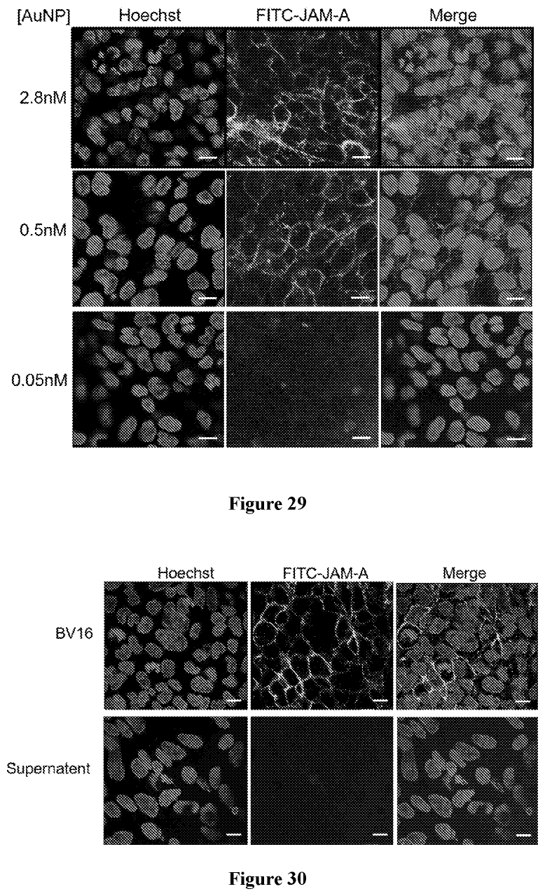

In order to determine the location of JAM-A-targeting AuNP, immunocytochemistry (ICC) staining of JAM-A was performed on hCMEC/D3 cells. The result shows well co-localization of AuNP-BV16 and tight junction (FIG. 2). The inventors observed obvious TJ targeting using 0.5 nM and 2.9 nM AuNP, but not 0.05 nM AuNP (FIG. 29). Thus, the inventors chose 0.5 nM AuNP to target JAM-A in hCMEC/D3 monolayer. In addition, the inventors checked the final supernatant from AuNP and BV16 conjugation to make sure there are no free antibody left in the AuNP-BV16 sample. The results showed free antibody had been removed after washing 3 times (FIG. 30).

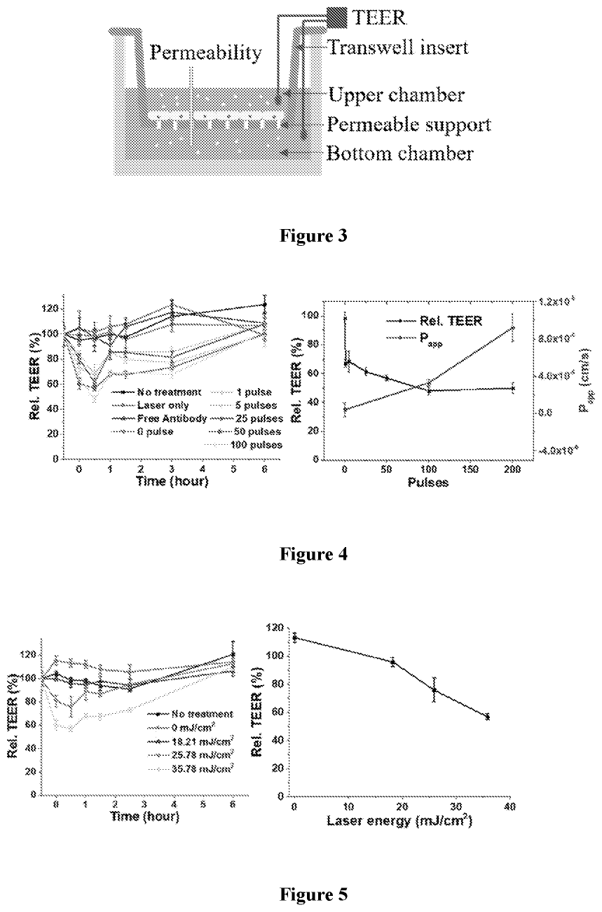

Temporary BBB opening in vitro. After confirming that AuNP can be targeted to TJ, the inventors applied picosecond (ps) laser pulses to create nanoscale force to temporarily open BBB. They measured the BBB opening by changes in TEER and permeability (FIG. 3).

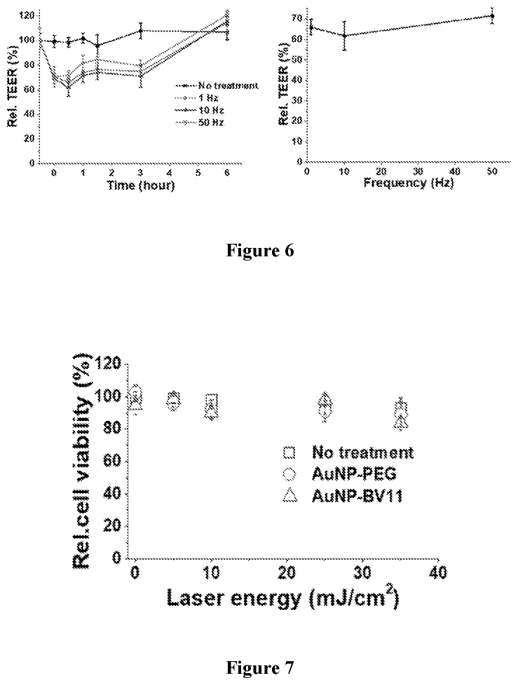

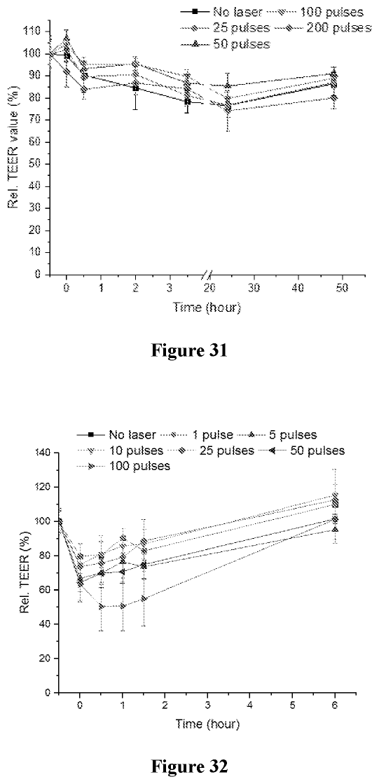

First, the inventors applied ps laser to hCMEC/D3 monolayer untreated by AuNP-BV16 and checked whether the highest laser energy cause cell damage indicated by TEER drop. The TEEER of hCMEC/D3 monolayer was not affected by laser irradiation (FIG. 31). Next, hCMEC/D3 monolayers were incubated with 0.5 nM 40 nm AuNP-BV16 at 37° C. for 0.5 hour and were then irradiated by ps laser. TEER values dropped by 30% for 1, 5, 10 and 25 pulses, and by 50% for 100 pulses (FIG. 4). The TEER of hCMEC/D3 recovered to 100% within 6 hours after laser irradiation. When varying laser pulse energy and keeping laser pulse number constant, the drop in TEER values increases with higher laser pulse energy, specifically by 25% at 25.78 mJ/cm² and by 40% at 35.78 mJ/cm² (FIG. 5). Laser pulse energy of 18.21 mJ/cm² did not lead to significant drop in TEER. The laser frequency did not cause significant change on TEER (FIG. 6).

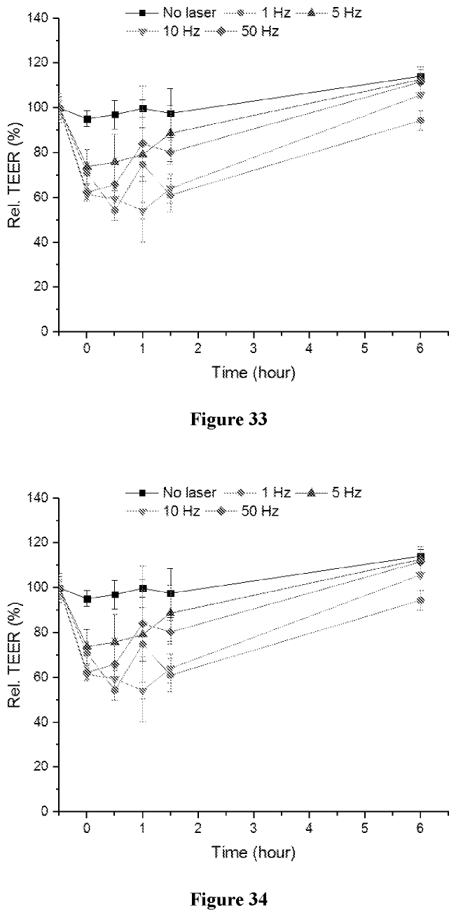

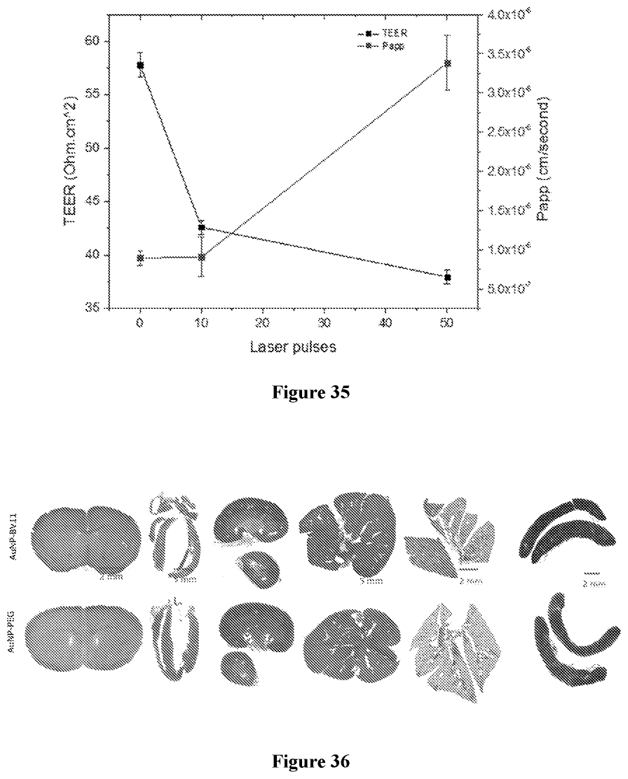

The inventors then measured the permeability change (Pₐₚₚ) by using 40 kDa FITC-dextran 40. The results show that Pₐₚₚ increased from 3.93±7.50×10⁻⁷ cm/s to 3.28±0.33×10⁻⁶ cm/s and 9.2±1.5×10⁻⁶ cm/s for 100 and 200 pulses, an increase by 8-fold and 23-fold, respectively. For comparison, TEER value dropped from 59.2±0.88 Ohm·cm² to 29.0±1.56 and 33.4±0.25 Ohm·cm² (FIG. 4). Investigation using a different gold nanoparticle size (60 nm) showed similar results (FIGS. 32-35).

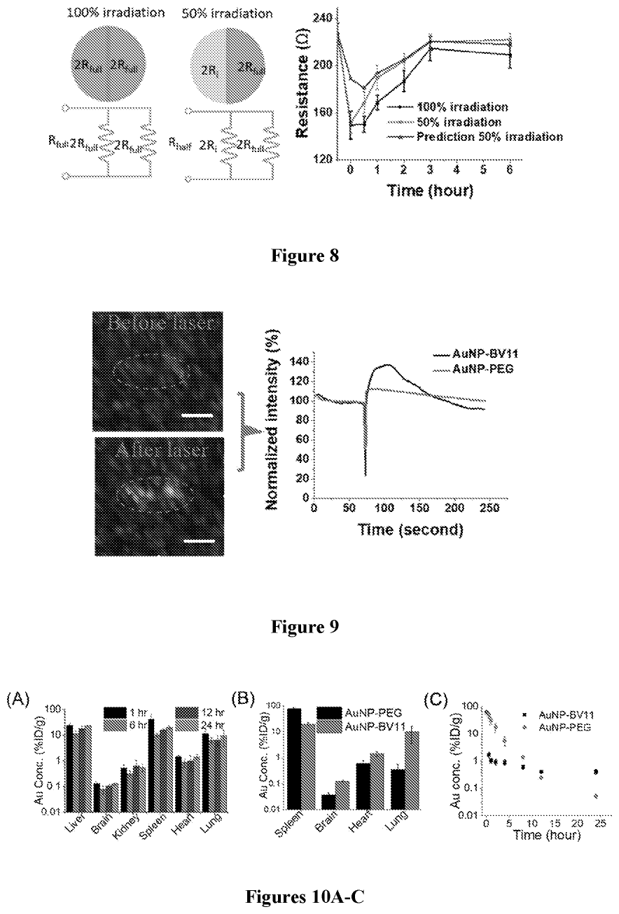

Next, the inventors tested the cell viability using MTT assay. The results show laser excitation of TJ-targeting AuNPs does not cause significant cell damage within 35 mJ/cm² and 25 pulses (FIG. 7). To understand the mechanism of the BBB opening, the inventors applied ps laser (35 mJ/cm², 10 pulses) to half of the insert micromembrane and measured the TEER which shows significant low than the prediction TEER value (FIG. 8). The results indicate BBB opening propagation happened. Finally, the inventors investigated the calcium (Ca²⁺) response to optical BBB opening. The result suggests that Ca²⁺ were transiently increased after ps laser application as indicated by the increase of fluo-4 intensity (FIG. 9), which potentially contribute to reversible BBB opening. Further study will test whether the Ca²⁺ signal propagate surrounding the laser stimulation area. Since G-actin happens polymerization and forms F-actin when environment cause stress to cells¹⁹, the inventors will test actin response to BBB opening.

The inventors also investigated nanosecond laser and TJ-targeting AuNP to manipulate BBB. First, they showed that the AuNP accumulation and laser irradiation does not affect cell proliferation (FIG. 41A). To characterize the changes in the BBB, the transendothelial electrical resistance (TEER) was measured (FIG. 41B). With a nanosecond laser, the TEER value dropped to 60% in 30 minutes (AuNP-BV16 group) while no significant drop was observed for AuNP-PEG groups and control groups (without AuNP incubation). Also, with the absence of laser irradiation, the inventors did not observe any obvious the TEER value drops for all three groups during experiments. After 6 hours, the TEER value for the MH group recovers to the original level comparable with the control groups. This is important since it indicates that nanosecond laser treatment leads to a reversible BBB opening. The endothelial barrier function was also assessed by measuring the permeability of macromolecules (FIG. 41C). The results show that the permeability of FITC-labeled dextran (FD) increased more than 25-times (molecular weight from 4 kDa to 70 kDa).

Biodistribution study of TJ-targeting AuNP. To investigate the in vivo biodistribution of TJ-targeting AuNPs (AuNP-BV11), the mice were administrated intravenously AuNP-BV11 at 36.9 μg/g, followed by ICP-MS analysis of gold concentration in major organs. Consistent with other nanomaterials²⁰, the inventors find major accumulation in liver and spleen (FIG. 10A). When compared with AuNP-PEG, AuNP-BV11 decreases spleen accumulation by 4-fold and increases the accumulation in brain by 4-fold, heart by 2.5-fold, and lung by 10-fold (FIG. 10B). Moreover, the pharmacokinetics study showed concentration of TJ-targeting AuNP in circulation system dropped much faster than non-targeting group (AuNP-PEG) within half of hour (FIG. 10C).

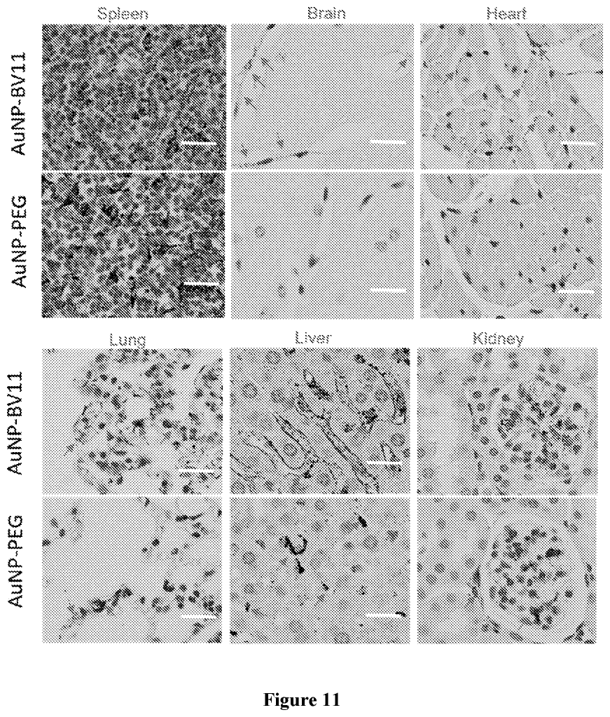

Next, the inventors performed silver enhancement staining to investigate the microscopic distribution of AuNP-BV11. Consistent with the results from ICP-MS analysis, there are less AuNP-BV11 in the spleen, more AuNP in brain, heart and lung when compared with AuNP-PEG (FIG. 11). AuNP-BV11 seems to be more dispersed in the liver than AuNP-PEG, while no significant difference was found in the kidney.

To measure in vivo toxicity of TJ-targeting AuNP, the inventors checked body weight of animals post-injection of 36.9 μg/g gold nanoparticle. The body weight of AuNP-BV11 group did not show significant change, compared with that of saline injection (FIG. 12A). That indicates the AuNP dose used is safe for animals, which was further confirmed by the histology analysis of major organs by comparing the nanoparticle injection versus control animal group (saline injection) (FIGS. 12B and 36).

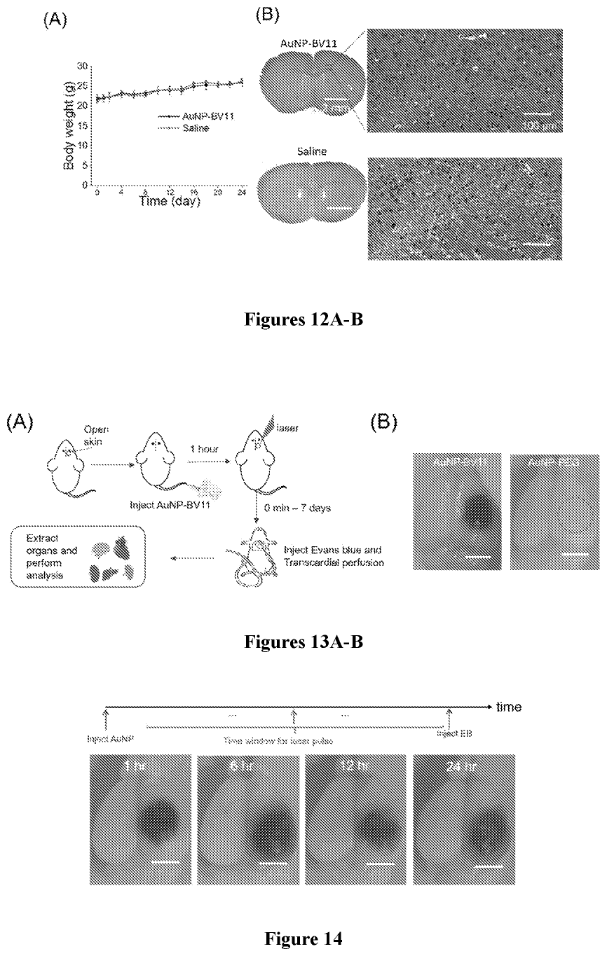

Transcranial optical BBB opening in vivo. The inventors injected C57BL/6 mice intravenously with AuNPs conjugated with anti-JAM-A antibody (AuNP-BV11) and non-targeting AuNPs (AuNP coated with polyethylene glycol, or AuNP-PEG), followed by intravenously injecting 100 μl 2% Evans blue in PBS (FIG. 13A). Laser pulse was then applied to the right hemisphere (532 nm, 1 pulse, 25 mJ/cm²) to test effect of TJ-targeting AuNPs on BBB opening. The result shows clear Evans blue (EB) extravasation in the mouse treated with TJ-targeting AuNPs (AuNP-BV11), while no EB extravasation in the mouse treated by non-targeting AuNPs (AuNP-PEG) (FIG. 13B). To test the timing of laser irradiation after AuNP administration, the laser pulse was applied at different time points (1, 6, 12 and 24 hours) post injection of AuNP-BV11. The results show consistent EB extravasation from 1 to 24 hours (FIG. 14).

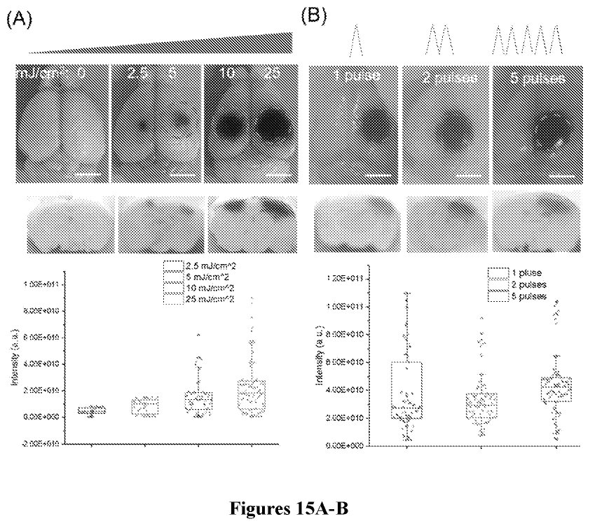

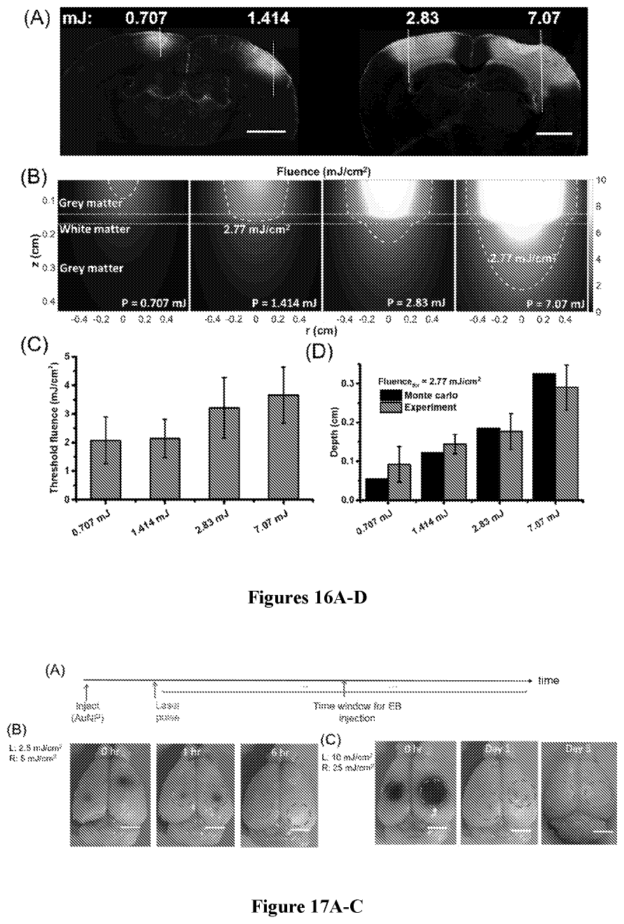

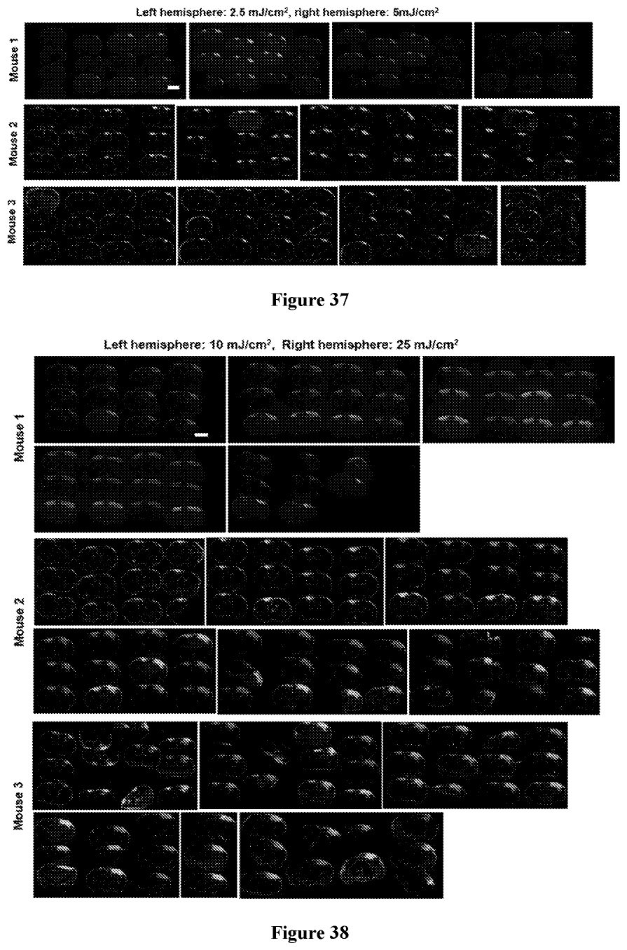

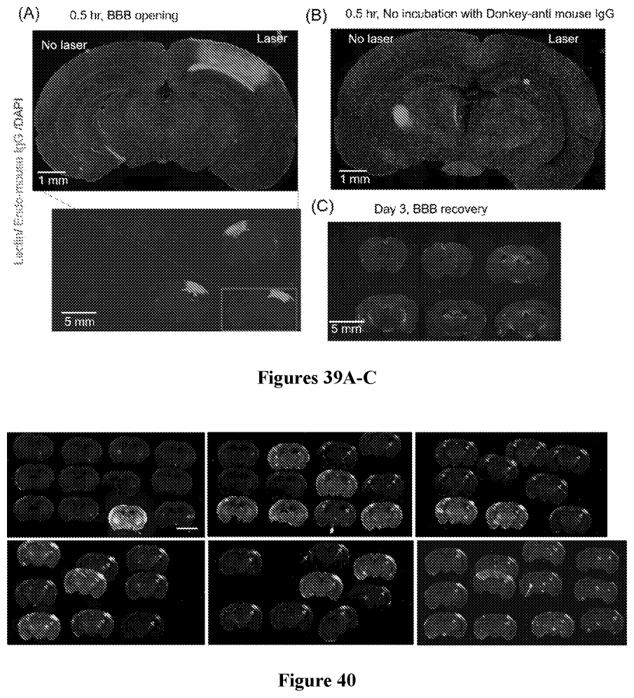

Next, the inventors tested the effects of laser pulse parameters (laser pulse number, energy) on the BBB opening. When increasing laser pulse energy (0, 2.5, 5, 10, and 25 mJ/cm²) and keeping the same laser pulse number (1 pulse), the inventors observed Evans blue extravasation starts at 2.5 mJ/cm² (FIG. 15A) and the Evans blue leakage is deeper with high energy. By varying laser pulse number (1, 2, 5 pulses) with same laser pulse energy at 25 mJ/cm², Evans blue extravasation increased with laser pulse number (FIG. 15B). In order to quantitatively analyze the depth of EB extravasation, the inventors performed cryosections processing of mouse brains and got EB fluorescent images (FIGS. 16A, 37 and 38) using slide scanner (Olympus VS120), followed by Fiji imageJ analysis. The results show the depth of EB penetration increases with laser pulse energy increases (FIGS. 16A and 16D). To study the light penetration of mouse brain tissues, the inventors perform the Monte Carlo simulation. The fluence distribution inside tissue for laser with different power was shown in FIG. 16B. As the power increases, the laser penetration depth also increases. The threshold fluence for BBB opening which is 2.77 mJ/cm². This threshold fluence was obtained by averaging the simulation results for all four cases which corresponds to experimental measurements (FIG. 16C). To confirm the laser fluence threshold, the inventors compared the depths measured in experiments and the depth where the fluence decay to threshold in the simulation. The depths obtained from two methods confirmed with each other which suggested that the Monte Carlo simulation is accurate and can be a good method to interpret the power dependence of BBB opening. (FIG. 16D).

To test the recovery of BBB, the inventors administrated Evans blue dye at 1 hour, 6 hours, 1 day and 3 days (FIG. 17A) post laser excitation TJ-targeting AuNPs. At low laser pulse energy (2.5 and 5 mJ/cm²), Evans blue extravasation disappears after 6 hours (FIG. 17B), while at intermediate and high laser pulse energies (10 and 25 mJ/cm²), the inventors observed less Evans blue extravasation at 6 hours and complete absence of Evans blue extravasation at day 3 (FIG. 17C).

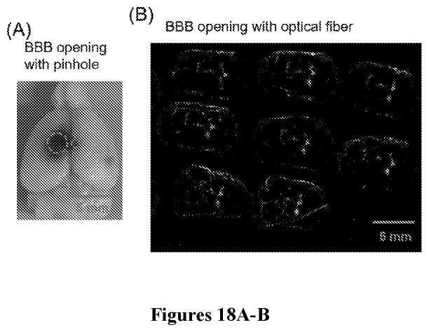

To further demonstrate the optical BBB opening can reach high spatial resolution, the inventors tested BBB opening by a pinhole laser (beam size of 0.8 mm and 2.5 mm) and optical fiber. The results suggested the BBB opening can be controlled optically with high spatial resolution (FIG. 18).

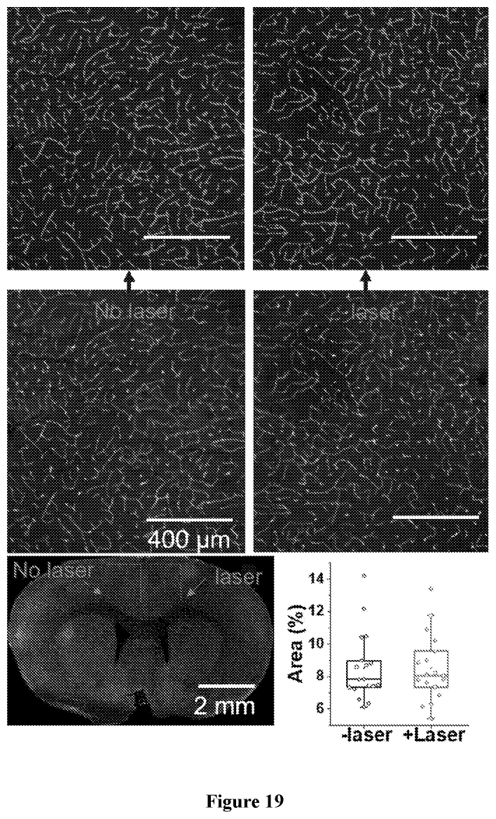

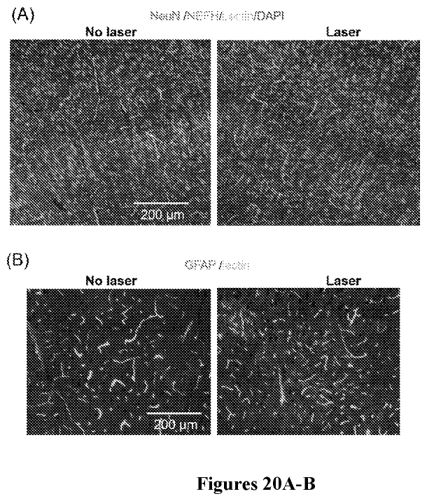

Brain vasculatures and brain cells response to temporary BBB opening. In order to analyze effect of BBB opening on the brain vasculatures, the inventors injected the tomato lectin to label vasculatures post laser irradiation with 25 mJ/cm². They performed the cryosection processing to get brain sections followed by slide scanning and then used Fiji imageJ to analyze the area fraction of brain blood vessels with and without laser treatment. The result shows there is no significant difference between laser and no laser treatment (FIG. 19).

The neuropathology analysis using IHC staining show, at day 3, the neurons appear normal indicated by no significant difference of the cell body (NeuN) and processes (NEFH) of neurons (FIG. 20A). The inventors observed a mild increase in active astrocytes (GFAP), which contribute to BBB recovery (FIG. 20B).

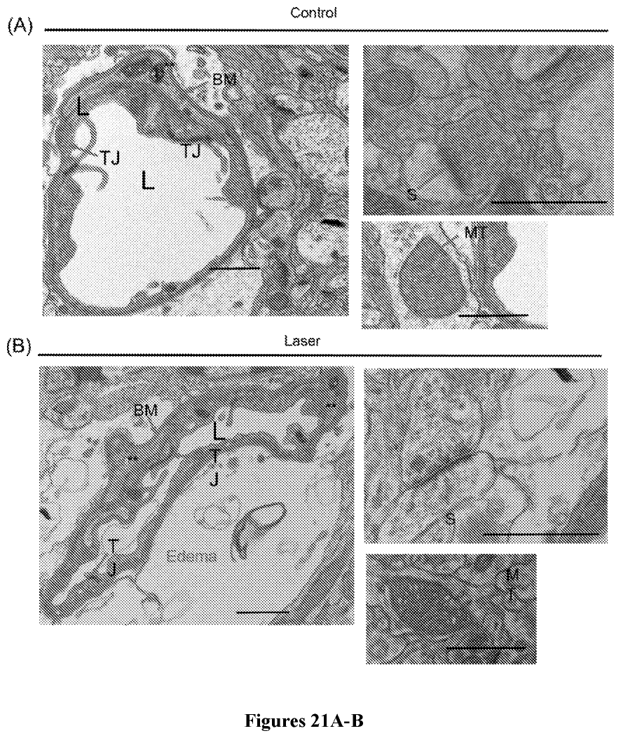

The inventors further perform the transmission electron microscopy (TEM) to check the brain microenvironment post laser irradiation. The results (FIG. 21) show, at 6 hours after laser excitation (5 mJ/cm², 1 pulse), the tight junction (TJ), basement membrane (BM), pericytes appear intact, indicating the BBB recovery. While edema was observed in the laser excitation areas indicating BBB opening, the synapse and mitochondrial are normal, which suggests minimal cytotoxicity.

BBB transient opening to deliver functional molecules to the brain. Here, the inventors demonstrate the delivery of functional molecules into the brain post laser application, such as anti-cancer drug (doxorubicin (Dox)) and antibodies (endogenous IgG: mouse; and exogenous IgG: human).

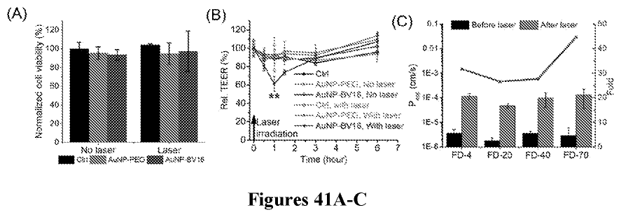

In order to deliver Dox and human IgG, the inventors inject Dox and human IgG instead of Evans blue dye. The IHC staining was used to stain the endogenous mouse IgG extravastion. The results show obvious Dox across the BBB (FIG. 22A). The results show fluorecent signal from mouse IgG (FIG. 22B, FIG. 39). and human IgG (FIG. 22C, FIG. 40) indicating IgG can cross the BBB after treated by TJ-targeting AuNP and ps laser.

### Example 3—Discussion

The BBB restricts the delivery most drug molecules into the brain by TJs that seal gap between adjacent endothelial cells with a low permeability and a high electrical resistance. Here the inventors report a novel method for temporary BBB opening. Specifically, TJ-targeting plasmonic gold nanoparticle open the BBB after excitation by ps pulse laser in vitro and in vivo. They attribute this to the nanoscale mechanical stress on the tight junction molecules. The biggest advantage of this optical BBB opening approach is the high optical resolution to investigate drug accumulation. This method can be used to investigate infiltrating gliomas especially in brain regions that does not show contrast enhancement under MRI, which beyond invasive and dangerous biopsies have been inaccessible to for drug penetration assessment. This work focuses on a critical scientific and clinical challenge of delivering drug to the brain by overcoming the blood-brain barrier. Optical BBB opening promises to create an entirely new paradigm to investigate significantly improved drug delivery efficiency to brain tumors and dramatically reduce side effects.

### Example 4—Exemplary Embodiments

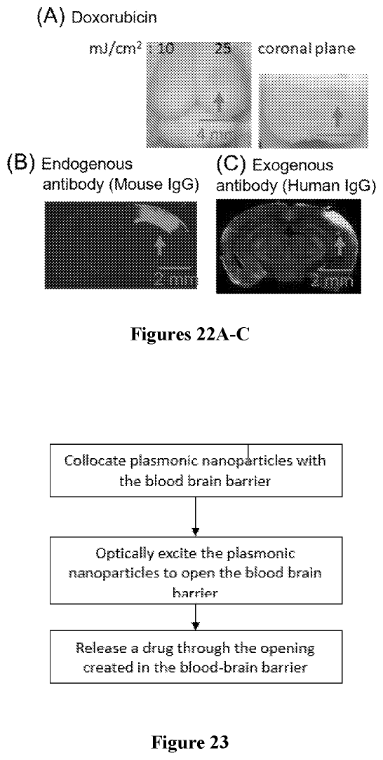

In an embodiment shown in FIG. 23, plasmonic nanoparticles are collocated with the blood-brain barrier, then the plasmonic nanoparticles are optically excited to open the blood-brain barrier. Once the blood-brain barrier is opened, then a drug is administered through the opening created in the blood-brain barrier.

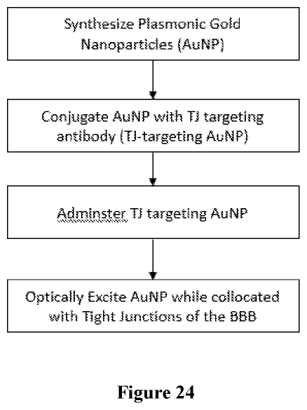

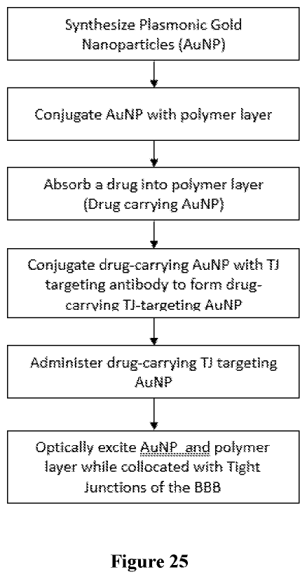

In another embodiment shown in FIG. 24, a process for optically opening the BBB is as follows: (1) synthesize AuNp, plasmonic gold nanoparticles, (2) modify the AuNP plasmonic gold nanoparticles by conjugating them with a TJ targeting antibody (for example, an anti-JAM-A antibody such as BV11 for mouse, and BV16 for human) to form a TJ-targeting AuLip nanoparticle solution, (3) intraveneously administer the TJ-targeting AuNP nanoparticle solution into patient wherein the AuNP particles are collocated to the tight junctions of the BBB and, (4) excite the AuNP plasmonic gold nanoparticles collocated near the BBB with short laser pulses (for example ps) at plasmonic absorption wavelength (for instance 532 nm for spherical AuNP, near-infrared for gold nanorod) and of sufficient optical power to perturb the TJ and endothelial cell, effectively opening the BBB.

In another embodiment shown in FIG. 25, a process for optically opening the BBB and delivering a drug behind the BBB is as follows: (1) synthesize AuNP, plasmonic gold nanoparticles, then (2) modify the AuNp plasmonic gold nanoparticles by conjugating a polymer layer around the AuNP plasmonic gold nanoparticles, (such as poly(vinylpyrroidone)), that can absorb anticancer drugs, (3) absorbing drug molecules (e.g., anti-cancer drug) into the polymer layer to form a drug-carrying AuNP, (3) conjugating the drug-carrying AuNP with a TJ targeting antibody (for example, an anti-JAM-A antibody such as BV11 for mouse, and BV16 for human) to form a drug-carrying TJ-targeting AuNP nanoparticle solution, (4) intraveneously administer the TJ-targeting AuNP nanoparticle solution into patient wherein the AuNP particles are collocated to the tight junctions of the BBB and, (5) excite the AuNP plasmonic gold nanoparticles and the polymer layer collocated near the BBB with short laser pulses (for example ps) at plasmonic absorption wavelength (for instance 532 nm for spherical AuNP, near-infrared 700-1000 nm for gold nanorod) and of sufficient optical power to perturb the tight junction and endothelial cells effectively creating an opening in the BBB and to release the drug molecules carried by the polymer layer through the opening.

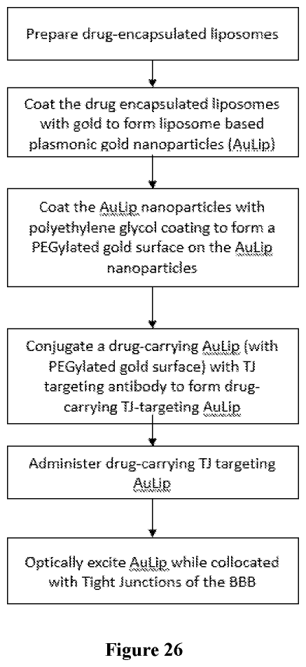

In another embodiment shown in FIG. 26 the process for utilizing AuLip to open the BBB and deliver drug molecules behind the BBB is as follows: (1) prepare drug-encapsulated liposomes, then (2) coat the drug encapsulated liposomes with gold to form plasmonic gold nanoparticle coated liposomes (AuLip) for local laser excitation, (for example, by a one-step reduction method reported earlier²¹⁻²³), (3) coat the AuLip nanoparticles with a polyethylene glycol coating to form a PEGylated gold surface on the AuLip nanoparticles, (4) Conjugate a TJ-targeting antibody to PEGylated gold surface via standard amine-coupling chemistry to form a TJ-targeting AuLip nanoparticle solution (BV11 for mouse, and BV16 for human^{24,25}), (5) intraveneously administer the TJ-targeting AuLip nanoparticle solution into patient wherein the AuLip particles are collocated to the tight junctions of the BBB and, (6) excite the AuLip plasmonic gold nanoparticles collocated at the tight junctions of the BBB with ps laser pulses in the near infrared (700-1000 nm) of sufficient optical power to perturb the tight junction and endothelial cells effectively creating an opening in the BBB and to release the drug molecules carried by the liposome through the opening²⁶.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
1. Tajes, M., et al. The blood-brain barrier: structure, function and therapeutic approaches to cross it. Mol Membr Biol 31, 152-167 (2014).
2. Abbott, N. J., Ronnback, L. & Hansson, E. Astrocyte-endothelial interactions at the blood-brain barrier. Nat Rev Neurosci 7, 41-53 (2006).
3. Pardridge, W. M. Drug transport across the blood-brain barrier. J Cereb Blood Flow Metab 32, 1959-1972 (2012).
4. Pardridge, W. M. The blood-brain barrier: bottleneck in brain drug development. NeuroRx 2, 3-14 (2005).
5. Doolittle, N. D., et al. Safety and efficacy of a multicenter study using intraarterial chemotherapy in conjunction with osmotic opening of the blood-brain barrier for the treatment of patients with malignant brain tumors. Cancer 88, 637-647 (2000).
6. Chu, P. C., et al. Focused Ultrasound-Induced Blood-Brain Barrier Opening: Association with Mechanical Index and Cavitation Index Analyzed by Dynamic Contrast-Enhanced Magnetic-Resonance Imaging. Sci Rep 6, 33264 (2016).
7. Kinoshita, M., McDannold, N., Jolesz, F. A. & Hynynen, K. Noninvasive localized delivery of Herceptin to the mouse brain by MRI-guided focused ultrasound-induced blood-brain barrier disruption. Proc Natl Acad Sci USA 103, 11719-11723 (2006).
8. Chen, H. & Konofagou, E. E. The size of blood-brain barrier opening induced by focused ultrasound is dictated by the acoustic pressure. J Cereb Blood Flow Metab 34, 1197-1204 (2014).
9. Gao, X., et al. Overcoming the blood-brain barrier for delivering drugs into the brain by using adenosine receptor nanoagonist. ACS Nano 8, 3678-3689 (2014).
10. Kovacs, Z. I., et al. Disrupting the blood-brain barrier by focused ultrasound induces sterile inflammation. Proc Natl Acad Sci USA 114, E75-E84 (2017).
11. Perrault, S. D. & Chan, W. C. Synthesis and surface modification of highly monodispersed, spherical gold nanoparticles of 50-200 nm. J Am Chem Soc 131, 17042-17043 (2009).
12. Chithrani, B. D., Ghazani, A. A. & Chan, W. C. Determining the size and shape dependence of gold nanoparticle uptake into mammalian cells. Nano Lett 6, 662-668 (2006).
13. Dai, Q., Walkey, C. & Chan, W. C. Polyethylene glycol backfilling mitigates the negative impact of the protein corona on nanoparticle cell targeting. Angew Chem Int Ed Engl 53, 5093-5096 (2014).
14. Haleh, S., Hirac, G. & Frederic, P. Optical properties of mice skull bone in the 455- to 705-nm range. J Biomed Opt 22, 10503 (2017).
15. Yaroslaysky, A. N., et al. Optical properties of selected native and coagulated human brain tissues in vitro in the visible and near infrared spectral range. Phys Med Biol 47, 2059-2073 (2002).
16. Custo, A., Wells, W. M., 3rd, Barnett, A. H., Hillman, E. M. & Boas, D. A. Effective scattering coefficient of the cerebral spinal fluid in adult head models for diffuse optical imaging. Appl Opt 45, 4747-4755 (2006).
17. Yang, G., Pan, F., Parkhurst, C. N., Grutzendler, J. & Gan, W. B. Thinned-skull cranial window technique for long-term imaging of the cortex in live mice. Nat Protoc 5, 201-208 (2010).
18. Wuest, D. M. & Lee, K. H. Optimization of endothelial cell growth in a murine in vitro blood-brain barrier model. Biotechnol J 7, 409-417 (2012).
19. Shi, Y., et al. Rapid endothelial cytoskeletal reorganization enables early blood-brain barrier disruption and long-term ischaemic reperfusion brain injury. Nat Commun 7, 10523 (2016).
20. Tsoi, K. M., et al. Mechanism of hard-nanomaterial clearance by the liver. Nat Mater 15, 1212-1221 (2016).
21. Troutman, T. S., Barton, J. K. & Romanowski, M. Biodegradable plasmon resonant nanoshells. Adv. Mater. 20, 2604-2608 (2008).
22. Sau, T. K. et al. Controlling loading and optical properties of gold nanoparticles on liposome membranes. Colloids Surfaces A Physicochem. Eng. Asp. 342, 92-96 (2009).
23. Rengan, A. K. et al. In vivo analysis of biodegradable liposome gold nanoparticles as efficient agents for photothermal therapy of cancer. Nano Lett. 15, 842-848 (2015).
24. Giannotta, M. et al. Targeting endothelial junctional adhesion molecule-A/EPAC/Rap-1 axis as a novel strategy to increase stem cell engraftment in dystrophic muscles. Embo Mol. Med. 6, 239-258 (2014).
25. Williams, L. a, Martin-Padura, I., Dejana, E., Hogg, N. & Simmons, D. L. Identification and characterisation of human Junctional Adhesion Molecule (JAM). Mol. Immunol. 36, 1175-1188 (1999).
26. Li, Xiuying et al. Ultrafast Near-Infrared Light-Triggered Intracellulas Uncaging to Probe Cell Signaling. Adv. Funct. Mater. 2017, 1605778

1. A method of generating nanoscale heating and photomechanical effects in vivo at the blood-brain barrier of a subject comprising:
(a) administering to said subject a light-absorbing particle, such as a plasmonic noble metal nanoparticle, wherein said light-absorbing particle comprises a targeting agent that directs said light-absorbing particle to the blood-brain barrier; and
(b) contacting said light-absorbing particle with a short pulse laser signal.

2. The method of claim 1, wherein said short pulse laser signal is a picosecond or nanosecond laser signal.

3. The method of claim 1, wherein said targeting agent is a receptor ligand.

4. The method of claim 1, wherein said target agent is a receptor ligand mimic.

5. The method of claim 1, wherein said targeting agent is an antibody.

6. The method of claim 3, wherein said targeting agent targets said light-absorbing particle to a blood-brain barrier tight junction, or endothelial membrane receptor such as transferrin.

7. The method of claim 6, wherein said targeting agent targets JAM-A, Claudin-5, ZO-1, or transferrin.

8. The method of claim 1, wherein said subject is a human.

9. The method of claim 1, wherein said subject is a non-human animal.

10. The method of claim 1, further comprising administering to said subject a therapeutic or diagnostic agent.

11. The method of claim 10, wherein said therapeutic or diagnostic agent is attached to said light-absorbing particle.

12. The method of claim 10, wherein said therapeutic or diagnostic agent is not attached to said light-absorbing particle.

13. The method of claim 1, wherein said laser signal is a near-infrared signal.

14. The method of claim 1, wherein said laser signal is a or visible signal.

15. The method of claim 1, wherein said light-absorbing particle is administered systemically, such as intravenously, intra-arterially, intrathecally, or retro-orbitally.

16. A method of delivering an agent in vivo to the brain of a subject comprising:
(a) administering to said subject a light-absorbing particle, wherein said light-absorbing particle comprises a targeting agent that directs said light-absorbing particle to the blood-brain barrier;
(b) administering to said subject an agent; and
(c) contacting said light-absorbing particle with a short pulse laser signal.

17. The method of claim 16, wherein said agent is a diagnostic agent.

18. The method of claim 17, wherein said diagnostic agent is a dye, a fluorophore, and chromophore, a contrast agent, or a radionuclide.

19. The method of claim 16, wherein said agent is a therapeutic agent.

20. The method of claim 19, wherein said therapeutic agent is an anti-cancer agent, such as a chemotherapeutic, a radiotherapeutic, an immunotherapeutic, or a gene therapeutic.

21. The method of claim 19, wherein said therapeutic agent is an antibiotic, an antifungal or an antiviral.

22. The method of claim 19, wherein said therapeutic agent is a neurotherapeutic agent, such as an anti-dementia drug, an anti-neurodegenerative disease drug, an anti-edema agent such as glyburide, or a gene therapy agent such as AAV.

23. The method of claim 16, wherein said targeting agent targets JAM-A, Claudin-5, ZO-1, or endothelial membrane receptor such as transferrin.

24. The method of claim 16, wherein said subject is a human.

25. The method of claim 16, wherein said subject is a non-human animal.

26. The method of claim 16, wherein said agent is attached to said light-absorbing particle.

27. The method of claim 16, wherein said agent is not attached to said light-absorbing particle.

28. The method of claim 16, wherein said laser signal is a near-infrared signal.

29. The method of claim 16, wherein said laser signal is a visible signal.

30. The method of claim 16, wherein said light-absorbing particle and/or said agent are administered systemically, such as intravenously, intra-arterially, intrathecally, or retro-orbitally.
